(19) United States
(12) Patent Application Publication
(10) Pub. No.: US 2013/0309246 A1
(43) Pub. Date: Nov. 21, 2013

(54) JAGGED1 AS A MARKER AND THERAPEUTIC TARGET FOR BREAST CANCER BONE METASTASIS

(71) Applicants: Yibin Kang, Princeton, NJ (US); Nilay Sethi, New Brunswick, NJ (US)

(72) Inventors: Yibin Kang, Princeton, NJ (US); Nilay Sethi, New Brunswick, NJ (US)

(73) Assignee: The Trustees of Princeton University, Princeton, NJ (US)

(21) Appl. No.: 13/982,688

(22) PCT Filed: Feb. 2, 2012
(86) PCT No.: PCT/US12/23655

§ 371 Date: Jul. 30, 2013

Related U.S. Application Data
(60) Provisional application No. 61/438,826, filed on Feb. 2, 2011.

(51) Int. Cl. G01N 33/68

(52) U.S. Cl. CPC G01N 33/6893
USPC 424/158.1; 435/7.92; 435/6.12; 514/44 A; 536/24.5; 536/23.1; 514/44 R

(57) ABSTRACT

A method of treating Jagged1 induced bone metastasis is provided. A method of analyzing patients with tumors insensitive to RANK targeting treatments, but may respond to Jagged1 or Notch targeting therapies is provided. A method of treating patients with Jagged1 induced bone metastasis is provided. A method of predicting the therapeutic of treating a cancer patient with bone metastasis is provided. A kit for treating patients with Jagged1 induced bone metastasis is provided. A kit for predicting the therapeutic outcome of treating a cancer patient with bone metastasis using RANKL inhibitors is provided.

This application claims the benefit of U.S. Provisional Application No. 61/438,826, filed Feb. 2, 2011, which is incorporated herein by reference as if fully set forth.

This invention was made with government support under Grant #W81XWH-06-1-0481 awarded by the Department of Defense, U.S. Army Medical Research & Material Command. The government has certain rights in this invention.

The sequence listing that was electronically filed with this application, titled “Sequence Listing,” created on Feb. 2, 2012 and having a file size of 25,598 bytes is incorporated by reference herein as if fully set forth.

### FIELD

The disclosure herein relates to the identification and treatment of breast cancer bone metastasis.

### BACKGROUND

Although classically known for its role in embryonic development, the Notch pathway is now being recognized for its aberrant activation in cancer. An oncogenic role for Notch was first discovered in T-cell acute lymphoblastic Leukemia (T-ALL) and then extended to other malignancies including lung, ovary, breast and skin cancers. Only recently has Notch signaling been associated with cancer progression; it was shown to regulate mediators of invasion in pancreatic cancer. The Notch Ligand Jagged1 is associated with cancer progression as it is overexpressed in poor prognosis prostate and breast cancer patients. However, the functional mechanism of the Notch pathway in breast cancer metastasis is poorly defined.

In breast cancer patients, certain pathways are aberrantly activated leading to not only primary tumor growth but also distant metastasis with particular tropism to the bone. The Notch pathway has been implicated in breast cancer primary tumor development, but has never been shown to contribute to breast cancer bone metastasis.

Bone metastasis affects over 70% of metastatic breast cancer with debilitating bone fractures, severe pain, nerve compression, and hypercalcemia. The development and outgrowth of these secondary lesions depends on the intricate cellular and molecular interactions between breast tumor cells and stromal cells of the bone microenvironment. In particular, the ability of tumor cells to disrupt the bone homeostatic balance maintained by two resident cell types, osteoclasts and osteoblasts, has been shown to drive bone destruction and metastatic tumor growth. Although several molecular contributors to bone metastasis have been identified, effective therapies still await a more comprehensive understanding of the complex molecular and cellular network of tumor-stromal interactions in bone metastasis.

### SUMMARY

In an aspect, the invention relates to a method for diagnosing an increased risk of breast cancer bone metastasis in a subject having breast cancer. The method includes obtaining a sample from the subject. The method also includes determining whether the sample has a Jagged1 high level expression marker. Presence of the Jagged1 high level expression marker in the sample indicates the increased risk of having breast cancer bone metastasis for the subject.

In an aspect, the invention relates to a method for diagnosing an increased risk of breast cancer bone metastasis in a subject having breast cancer. The method includes obtaining a sample from the subject. The method also includes determining whether the sample has a Jagged1 high level expression marker. The presence of the Jagged1 high level expression marker in the sample indicates the increased risk of having breast cancer bone metastasis for the subject. The method also includes diagnosing the subject as having an increased risk of breast cancer bone metastasis upon determining the presence of the Jagged1 high level expression marker in the sample. The method may also include diagnosing the subject as having decreased sensitivity to RANK or RANKL targeting treatments upon determining the presence of the Jagged1 high level expression marker in the sample. The method may also include diagnosing the subject as having increased sensitivity to NOTCH targeting treatments upon determining the presence of the Jagged1 high level expression marker in the sample. The method may also include diagnosing the subject as having increased sensitivity to Jagged1 targeting treatments against breast cancer bone metastasis upon determining the presence of the Jagged1 high level expression marker in the sample.

In an aspect, the invention relates to a method of treating a breast cancer patient. The method includes administering to the breast cancer patient at least one therapy selected from the group consisting of Notch targeting treatments and Jagged1 targeting treatments. The administering occurs after a determination of a presence of a Jagged1 high level expression marker in a sample from the breast cancer patient.

In an aspect, the invention relates to a composition comprising at least one agent selected from the group consisting of a Jagged1 activity down regulator, a Jagged1 gene expression down regulator, an RNAi molecule that has a nucleotide sequence complementary to at least a portion of Jagged1 mRNA, and a DNA encoding the RNAi molecule that has a nucleotide sequence complementary to at least a portion of Jagged1 mRNA. The composition may also include a pharmaceutically acceptable carrier.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIGS. 1A-1D illustrate the relapse rate in patients with high or low expression of JAG1, NOTCH1 and HES1. FIG. 1A shows the Kaplan-Meier relapse-free survival curve of patients from the Wang data set (Wang et al., 2005, which is incorporated herein by reference as if fully set forth) with either low or high expression of JAG1. FIGS. 1B-1D show Kaplan-Meier relapse-free survival curves of patients from the Wang et al. data set (Wang et al., 2005, which is incorporated herein by reference as if fully set forth) with either low or high expression of NOTCH1 and HES1 (two probes).

FIGS. 2A-2D illustrate the bone metastasis-free survival curve in patients with high or low expression of JAG1 of indicated Notch receptor genes. FIG. 2A shows the bone metastasis-free survival curve of the Minn data set (Minn et al., 2005, which is incorporated herein by reference as if fully set forth) with either low or high expression of JAG1. FIGS. 2B-2D show Kaplan-Meier bone metastasis-free survival curves of patients from the Minn et al. data set (Minn et al., 2005, which is incorporated herein by reference as if fully set forth) with either low or high expression of indicated Notch receptor genes.

FIG. 3A illustrates a western blot analysis showing JAGGED1 (JAG1) protein levels in the control and JAGGED1 knockdown (KD) for sublines SCP2 and 1833.

FIG. 3B illustrates mRNA expression of JAG1 in the MDA231 cell line and its derivative sublines with distinct bone metastasis properties using qRT-PCR.

FIG. 4 illustrates qRT-PCR expression levels of Notch target genes Hey1 and Hes1 in the stromal compartment of control of JAG1 OE metastatic lesions using mouse-specific primers. Data represent average±SEM.

FIG. 5A illustrates mRNA expression of JAG1 in response to TGFβ treatment in the weakly (left) and strongly (right) bone-metastatic MDA231 sublines using previously reported microarray expression profiling data (Kang et al., 2003, which is incorporated herein by reference as if fully set forth).

FIGS. 5B and 5C illustrate JAGGED1 mRNA and protein levels in response to a time-course of TGFβ treatment in SCP28 cell line in the presence or absence of a TGFβ Receptor 1 kinase inhibitor (EMD616451) using qRT-PCR (5B) and western blot (5C) analysis.

FIG. 6A illustrates JAGGED1 mRNA levels in the tumor compartment of bone metastasis of mice treated with either a solvent control (n=7) or a TGFβ Receptor 1 kinase inhibitor (LY2109761, Eli Lilly) (n=4) using human-species specific qRT-PCR (Korpal et al., 2009, which is incorporated herein by reference as if fully set forth).

FIG. 6B illustrates qRT-PCR mRNA expression levels of JAG1 in the SCP28 cell line with inducible (Tet-off) SMAD4 expression (Korpal et al., 2009, which is incorporated herein by reference as if fully set forth) under the indicated TGFβ and doxycycline treatment conditions. Data represent average±SD.

FIG. 6C illustrates western blot analysis of JAGGED1 protein levels in the control or SMAD4-KD SCP28 cell lines (Kang et al., 2005, which is incorporated herein by reference as if fully set forth) in the presence or absence of TGFβ.

FIG. 6D illustrates western blot analysis of JAGGED1 protein levels in the control and JAG1-KD 1833 and SCP2 sublines in the presence and absence of TGFβ.

FIG. 7A illustrates coculture between control or JAG1 overexpressing (OE) SCP28 tumor cells and MC3T3-E1 osteoblasts transfected with a Notch reporter and treated with DMSO or MRK-003.

FIG. 7B illustrates qRT-PCR mRNA expression levels of indicated Notch target genes and TGFβ1 in MC3T3-E1 osteoblasts that were FACS-separated from cocultures in each experimental group. *p<0.05, **p<0.01, ***p<0.001.

FIG. 8A illustrates representative images of cocultures from each experimental group. White boxes indicate areas shown at higher magnification in the middle row. Tumor cells cultured alone are shown in the bottom row. Scale bar, 200 μM.

FIG. 8B illustrates quantification of tumor cells from cocultures with MC3T3-E1 from each experimental group by luciferase assay. *p=0.01, **p=0.007.

FIG. 8C illustrates the diameter of tumor colonies from cocultures of each experimental group. ***p<10⁻⁷.

FIG. 9A illustrates quantification of control or JAG1 OE tumor cells cocultured with MC3T3-E1 cells and treated with DMSO, 1 μM, or 5 μM MRK-003 by luciferase assay. *p<0.05.

FIG. 9B illustrates quantification of tumor cells cultured alone.

FIG. 9C illustrates cell cycle profiling of control and JAGGED1-overexpressing SCP28 tumor cells treated with MRK-003 or DMSO.

FIG. 10A illustrates qRT-PCR mRNA expression of several Notch target genes or bone related genes (Runx2, Osx and TGFβ1) in primary bone marrow osteoblasts that were cocultured with either SCP28 vector control or JAG1 OE tumor cells using mouse-specific primers. Data represent average±SD.

FIG. 10B illustrates quantification of tumor cells from cocultures with primary bone marrow derived osteoblasts from each experimental group by luficerase assay. Data represent average±SD. *p=0.01, **p<0.006.

FIG. 11A illustrates quantification of indicated tumor cells cocultured with MC3T3-E1 cells that were treated with Rbpj siRNAs (SEQ ID NO: 6 and SEQ ID NO: 7) by luciferase assay. *p<0.05.

FIG. 11B illustrates a heat map depicting microarray gene expression profiling of MC3T3-E1 osteoblasts that were FACS-separated from cocultures of each experimental group.

FIG. 12A illustrates qRT-PCR mRNA expression of Hey1 in MC3T3-E1 osteoblasts treated with control or Hey1 siRNAs (SEQ ID NO: 8 and SEQ ID NO: 9) and cultured in 12-well plates coated with either Fc control or recombinant JAG1 protein. Data represent average±SD. Student's t-test *p<0.05.

FIG. 12B illustrates quantification of indicated tumor cells cocultured with MC3T3-E1 cells that were treated with Hey1 siRNAs (SEQ ID NO: 8 and SEQ ID NO: 9) by luciferase assay. **p<0.005.

FIG. 13A illustrates a list of genes with expression levels greater than 3-fold in osteoblasts cocultured with JAG1 OE tumor cells relative to controls.

FIG. 13B illustrates quantification of IL-6 levels in conditioned media of control or JAG1 OE tumor cells cultured alone or cocultured with MC3T3-E1 cells in the presence of DMSO, 1 μM, or 5 μM MRK-003 using ELISA. ***p<1×10⁻⁵.

FIG. 13C illustrates ELISA quantification of IL-6 levels in conditioned media of indicated tumor cells cocultured with MC3T3-E1 cells treated with Rbpj siRNAs. **p<0.0005, ***p<1×10⁻⁴.

FIG. 13D illustrates quantification of IL-6 levels in conditioned media of indicated tumor cells cocultured with MC3T3-E1 cells treated with Hey1 siRNAs (SEQ ID NO: 8 and SEQ ID NO: 9) using ELISA. ***p<0.0005.

FIG. 13E illustrates qRT-PCR mRNA expression of IL-6 in flow cytometry-separated MC3T3-E1 osteoblasts from cocultures with control or JAG1 OE tumor cells in the presence of either DMSO control or 1 μM MRK-003. Data represent average±SD.

FIG. 13F illustrates qRT-PCR mRNA expression of IL-6 in MC3T3-E1 osteoblasts treated with control or Hey1 siRNAs (SEQ ID NO: 8 and SEQ ID NO: 9) and cultured in 12-well plates coated with either Fc control or recombinant JAGGED1 protein. Data represent average±SD. Student's t-test **p<0.0001.

FIG. 14A illustrates quantification of indicated tumor cells cocultured with MC3T3-E1 cells and treated with IgG, 0.5 μg/ml, or 1.0 μg/ml anti-mouse IL-6 by luciferase assay. *p<0.05, **p=0.007.

FIG. 14B illustrates quantification of indicated tumor cells cocultured with MC3T3-E1 cells and treated with PBS, 10 ng/ml, or 100 ng/ml hIL-6 by luciferase assay. *p<0.05, ***p<1 3 10⁻⁵.

FIG. 15A illustrates quantification of TRAP+ osteoclasts from TRAP staining of cocultures of control or JAG1 OE tumor cells with pre-osteoclast Raw 264.7 cells treated with DMSO or 1 μM MRK-003 immediately after seeding.

FIG. 15B illustrates qRT-PCR mRNA expression levels of mouse Apc5 (encoding mouse TRAP) from TRAP staining of cocultures of control or JAG1 OE tumor cells with pre-osteoclast Raw 264.7 cells treated with DMSO or 1 mM MRK-003 immediately after seeding (Early) or 2 days after seeding (Late).

FIG. 15C illustrates the diameter of TRAP+ osteoclasts from TRAP staining of cocultures of control or JAG1 OE tumor cells with pre-osteoclast Raw 264.7 cells treated with DMSO or 1 mM MRK-003 immediately after seeding.

FIGS. 16A-16E illustrate bone metastasis studies in mice. FIG. 16A shows normalized BLI signals of bone metastasis in mice (n=10) that have been injected with SCP2 cells and treated with vehicle or MRK-003. *p<0.05, **p<0.005. FIG. 16B shows the Kaplan-Meier bone metastasis-free survival curve of the mice. FIG. 16C shows the quantification of total and hindlimb bone lesions in vehicle or MRK003-treated mice. *p<0.05. FIG. 16D shows the quantification of radiographic osteolytic lesion area of hindlimbs of mice from each experimental group. FIG. 16E shows quantification of TRAP+ osteoclasts along the bone-tumor interface of metastases of mice from each experimental group.

FIGS. 17A-17D illustrate further metastasis studies in mice. FIG. 17A shows qRT-PCR mRNA expression of Notch target genes and mouse IL-6 in the stromal compartment of bone metastasis from vehicle or MRK-003-treated mice using mouse-specific primers. *p<0.005, **p<0.001. FIG. 17B shows Kaplan-Meier bone metastasis-free survival curve of mice from each experimental group over time (left), log rank p=0.032 and the normalized BLI signals of bone metastasis in mice inoculated with control or JAG1 OE tumor cells and treated with vehicle or MRK-003 (right). *p<0.05, **p<0.01 based on repeated-measures ANOVA. FIG. 17C shows quantification of radiographic osteolytic lesion area of mice hindlimbs from each experimental group. *p<0.05 by Student's t test. FIG. 17D shows quantification of TRAP+ osteoclasts along the bone-tumor interface of metastases from each experimental group. **p<0.005, ***p<1 3 10_{—}4 by Student's t test.

FIGS. 18A-18B illustrate Jagged1 KD western blots.

### DETAILED DESCRIPTION OF EMBODIMENTS

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. The phrase “at least one” followed by a list of two or more items, such as A, B, or C, means any individual one of A, B or C as well as any combination thereof.

The results herein are the first to show that Jagged1 alone can activate osteoclast differentiation without RANKL or with a minimal amount of RANKL. The results herein are the first to show that Jagged1 operates in a parallel pathway to osteoclast differentiation compared to the pathway activated by RANKL.

Embodiments include diagnostic methods, methods of treatment and kits based on the findings herein for the diagnosis, treatment or prevention of breast cancer metastasis to bone.

Embodiments include methods of treating bone metastasis. Embodiments include methods of treating breast cancer bone metastasis induced by Jagged1 in patients. The patient may be human. The methods include a step of administering an inhibitor of Jagged1, an inhibitor of IL-6, an inhibitor of IL-6R or an inhibitor of an IL-6R downstream signal transducer. These inhibitors include without limitation an antibody or fragments thereof against Jagged1, a monoclonal antibody or fragments thereof against Jagged1, an antibody or monoclonal antibody (or fragments of either) against IL-6, an antibody or monoclonal antibody (or fragments of either) against IL-6R and small molecular inhibitors of IL-6R downstream signal transducers. These inhibitors include without limitation small molecular inhibitors of the IL-6R downstream signal transducer Jak2. Small molecular inhibitors of IL-6R downstream signal transducers that may be administered in a method for treating herein include but are not limited to Ruxolitinib.

Embodiments include cancer treating drugs that may be used for treating breast cancer bone metastasis. Embodiments include cancer treating drugs that may be used to treat breast cancer bone metastasis induced by Jagged1 in patients. The patient may be human. The cancer treating drugs may be an inhibitor of Jagged1, an inhibitor of IL-6, an inhibitor of IL-6R or an inhibitor of an IL-6R downstream signal transducer. These inhibitors include without limitation an antibody or fragments thereof against Jagged1, a monoclonal antibody or fragments thereof against Jagged1, an antibody or monoclonal antibody (or fragments of either) against IL-6, an antibody or monoclonal antibody (or fragments of either) against IL-6R and small molecular inhibitors of IL-6R downstream signal transducers. These inhibitors include without limitation small molecular inhibitors of the IL-6R downstream signal transducer Jak2. The cancer treating drugs may be any one or more agent described herein that decreases Jagged1 or IL-6 expression or inhibits the activity thereof.

Embodiments include a pharmaceutical composition including any of the cancer treating drugs herein and a pharmaceutically acceptable carrier. The pharmaceutically acceptable carrier may include at least one of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.), or phosphate buffered saline (PBS).

The route for administering a drug or pharmaceutical composition may be by any route. The route of administration may be any one or more route including but not limited to oral, injection, topical, enteral, rectal, gastrointestinal, sublingual, sublabial, buccal, epidural, intracerebral, intracerebroventricular, intracisternal, epicutaneous, intradermal, subcutaneous, nasal, intravenous, intraarterial, intramuscular, intracardiac, intraosseous, intrathecal, intraperitoneal, intravesical, intravitreal, intracavernous, intravaginal, intrauterine, extra-amniotic, transdermal, intratumoral, and transmucosal.

Embodiments include a method of analyzing tumors. Embodiments include a method of analyzing tumors using at least one of Jagged1 or IL-6 as a biomarker, tumor marker or a serum marker. As used herein, “tumor marker” means a biomarker that is searched for in a tumor or tumor sample. As used herein, “serum marker” means a biomarker that is searched for in serum or serum samples. The method may include at least one of diagnosing a breast cancer patient as having an increased risk of breast cancer bone metastasis, lower sensitivity to RANK or RANKL targeting treatments, higher sensitivity to Jagged1 targeting treatments, or higher sensitivity to Notch targeting treatments upon a detection of a high level of at least one of Jagged1 or IL-6 in a breast cancer patient tumor or tumor sample. Lower sensitivity to RANK or RANKL targeting treatments may mean the breast cancer patient is unlikely to respond to current methods of treatment with RANK or RANKL targeting treatments. Unlikely to respond may mean that the patient is less likely to respond to the current methods than a patient with tumors lacking a high level of at least one of Jagged1 or IL-6. Embodiments include analyzing tumors to determine if a patient is unlikely to respond to current methods of treatment using denosumab, which is a monoclonal antibody against RANKL. Higher sensitivity to Jagged1 or Notch targeting treatments may mean the patient is more likely to respond to Jagged1 or Notch targeting therapies than a patient with tumors lacking a high level of at least one of Jagged1 or IL-6.

Embodiments include a method of treating a cancer patient comprising obtaining a sample from a patient, analyzing the sample to determine the existence of one or more indications associated with Jagged1-induced bone metastasis and administering the bone metastasis therapeutic agent to the patient upon a positive determination that the patient has at least one of the one or more indications associated with Jagged1 induction of bone metastasis. These indications include without limitation a Jagged1 biomarker, tumor marker or serum marker or an IL-6 biomarker, tumor marker or serum marker. The biomarker, tumor marker or serum marker may be the presence of elevated levels of Jagged1, IL-6, IL-6R, or IL-6R downstream signal transducers (which include without limitation Jak2), a mutation in one or more of these molecules or a genetic and epigenetic alteration leading to altered expression levels of one or more o these molecules. For example, a mutation leading to increased levels of Jagged1 may be an indication. The therapeutic agents include without limitation Notch targeting therapeutics, including gamma-secretase inhibitor (GSI). The therapeutic agents include without limitation Jagged1 targeting therapies, including RNAi molecules that inhibit Jagged1; an inhibitor of one or more of IL-6, IL-6R; or IL-6R downstream signal transducers; a monoclonal antibody against Jagged1, Notch receptors, IL-6, or IL-6R, or a small molecular inhibitor against IL-6R downstream signal transducers. These IL-6R downstream signal transducers include without limitation Jak2. The therapeutic agents include without limitation a receptor 1 kinase inhibitor. The therapeutic agents include without limitation MRK-003.

Embodiments include a method of predicting the therapeutic outcome of treating a cancer patient with a bone metastasis therapeutic agent comprising obtaining a sample from the patient and analyzing the sample to determine the existence of one or more indications associated with Jagged1 induction of bone metastasis. These indications include without limitation a Jagged1 biomarker, tumor marker or serum marker or an IL-6 biomarker, tumor marker or serum marker. The biomarker, tumor marker or serum marker may be the presence of a high expression level of Jagged1, IL-6, IL-6R, or IL-6R downstream signal transducers, which include without limitation Jak2. An indication may be a mutation of Jagged1, or an epigenetic change in the Jagged1 promoter.

Embodiments include a kit for treating a cancer patient comprising a detecting agent of one or more indications associated with Jagged1 induction of bone metastasis and a bone metastasis therapeutic agent. The detecting agent may be any compound capable of detecting the level of at least one of Jagged1 DNA or variants thereof, Jagged1 RNA or variants thereof, Jagged1 protein or variants thereof, IL-6 DNA or variants thereof, IL-6 RNA or variants thereof, IL-6 protein or variants thereof. The detecting agents contemplated include but are not limited to compounds used in DNA or RNA detection or quantification including northern blot, RT-PCR, SAGE, RNA-Seq (e.g., oligonucleotides complementary to nucleic acids coding for or involved in the regulation of Jagged1, IL-6, IL-6R, or IL-6R downstream signal transducers or variants of any of the foregoing, or other nucleic acid detection reagents); compounds used in protein quantification including western blot (e.g., antibodies that bind Jagged1, IL-6, IL-6R, or IL-6R downstream signal transducers or variants of any of the foregoing). The detecting agent may be any agent described herein for detecting Jagged1 DNA or variants thereof, Jagged1 RNA or variants thereof, Jagged1 protein or variants thereof, IL-6 DNA or variants thereof, IL-6 RNA or variants thereof, or IL-6 protein or variants thereof. The indications include without limitation a Jagged1 biomarker, tumor marker or serum marker or an IL-6 biomarker, tumor marker or serum marker. The therapeutic agents include without limitation Notch targeting therapeutics, including gamma-secretase inhibitor (GSI). The therapeutic agents include without limitation Jagged1 targeting therapeutics, including an RNAi molecule that inhibits Jagged1 or Notch, an antibody or fragment thereof against Jagged1 or Notch, a monoclonal antibody or fragment thereof against Jagged1 or Notch, an inhibitor of one or more of IL-6, IL-6R or IL-6R downstream signal transducers, an antibody or fragment thereof against IL-6, a monoclonal antibody or fragment thereof against IL-6, an antibody or fragment thereof against IL-6R, a monoclonal antibody or fragment thereof against IL-6R, or a small molecular inhibitor against Jagged1, IL-6, IL-6R or IL-6R downstream signal transducers. The IL-6R downstream signal transducers include without limitation Jak2.

Embodiments include a kit for predicting the outcome of treating a cancer patient, preferably a breast cancer patient, with a bone metastasis therapeutic agent comprising a detecting agent of one or more indications associated with Jagged1 induction of bone metastasis. The detecting agent includes any compound capable of detecting Jagged1 or IL-6 DNA, RNA or protein levels or variants of any of the foregoing. These include but are not limited to compounds used in DNA or RNA detection and quantification including northern blot, RT-PCR, SAGE, RNA-Seq; compounds used in protein quantification including western blot, ELISA, IHC and FACS. These indications include without limitation a Jagged1 biomarker, tumor marker or serum marker or an IL-6 biomarker, tumor marker or serum marker.

Embodiments include a method to treat breast cancer bone metastasis by targeting an important pathway (Jagged1/Notch signaling) in the tumor stromal microenvironment that is activated by tumor cells overexpressing Jagged1. Embodiments also present a novel method to use Jagged1 as a biomarker to identify breast cancer patients with high risk of at least one of relapse, metastasis, or bone metastasis. Jagged1 may also serve as a diagnostic marker to identify patients whose bone metastasis may be refractory to currently available RANK targeting treatments with Denosumab (Amgen). Those patients may instead benefit from Jagged1/Notch targeting treatments, and methods herein include providing such a diagnosis or a method of treating based on the same.

The methods herein can be used to reduce morbidity and mortality resulting from osteolytic bone metastasis of breast cancer. Furthermore, Jagged1 overexpression and Notch signaling activity in tumor stroma can be used as a poor-prognostic marker for higher risk of bone metastasis and a predictive marker to identify breast cancer patients who may be non-responsive to RANK or RANKL targeting treatment but are likely to benefit from Jagged1/Notch targeting treatments.

An embodiment includes a method for diagnosing an increased risk of breast cancer bone metastasis in a subject having breast cancer. The method may include obtaining a sample from the subject. The method may include determining whether the sample has a Jagged1 high level expression marker. The presence of the Jagged1 high level expression marker in the sample indicates the increased risk of having breast cancer bone metastasis for the subject.

The Jagged1 high level expression marker may be a level of Jagged1 in the sample that is higher than the level of Jagged1 found in normal tissue of the same type as the sample. The Jagged1 high level expression marker may be a level of Jagged1 in the sample that is higher than the level of Jagged1 found in tissue of the same type as the sample but from an individual lacking breast cancer metastasis to bone. The Jagged1 high level expression marker may be a level of Jagged1 in the sample that is higher than the level of Jagged1 found in tissue of the same type as the sample but from an individual having breast cancer but lacking breast cancer metastasis to bone. The Jagged1 high level expression marker may be a level of Jagged1 in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of Jagged1 found in normal tissue of the same type as the sample. The Jagged1 high level expression marker may be a level of Jagged1 in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of Jagged1 found in tissue of the same type as the sample but from an individual lacking breast cancer metastasis to bone. The Jagged1 high level expression marker may be a level of Jagged1 in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of Jagged1 found in tissue of the same type as the sample but from an individual having breast cancer but lacking breast cancer metastasis to bone. The Jagged1 high level expression marker may be a level of Jagged1 in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of Jagged1 found in a control sample.

The Jagged1 high level expression marker may be a level of Jagged1 mRNA in the sample that is higher than the level of Jagged1 mRNA found in normal tissue of the same type as the sample. The Jagged1 high level expression marker may be a level of Jagged1 mRNA in the sample that is higher than the level of Jagged1 mRNA found in tissue of the same type as the sample but from an individual lacking breast cancer metastasis to bone. The Jagged1 high level expression marker may be a level of Jagged1 mRNA in the sample that is higher than the level of Jagged1 mRNA found in tissue of the same type as the sample but from an individual having breast cancer but lacking breast cancer metastasis to bone. The Jagged1 high level expression marker may be a level of Jagged1 mRNA in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of Jagged1 mRNA found in normal tissue of the same type as the sample. The Jagged1 high level expression marker may be a level of Jagged1 mRNA in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of Jagged1 mRNA found in tissue of the same type as the sample but from an individual lacking breast cancer metastasis to bone. The Jagged1 high level expression marker may be a level of Jagged1 mRNA in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of Jagged1 mRNA found in tissue of the same type as the sample but from an individual having breast cancer but lacking breast cancer metastasis to bone. The Jagged1 high level expression marker may be a level of Jagged1 mRNA in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of Jagged1 mRNA found in a control sample.

The Jagged1 high level expression marker may be a level of IL-6 in the sample that is higher than the level of IL-6 found in normal tissue of the same type as the sample. The Jagged1 high level expression marker may be a level of IL-6 in the sample that is higher than the level of IL-6 found in tissue of the same type as the sample but from an individual lacking breast cancer metastasis to bone. The Jagged1 high level expression marker may be a level of IL-6 in the sample that is higher than the level of IL-6 found in tissue of the same type as the sample but from an individual having breast cancer but lacking breast cancer metastasis to bone. The Jagged1 high level expression marker may be a level of IL-6 in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of IL-6 found in normal tissue of the same type as the sample. The Jagged1 high level expression marker may be a level of IL-6 in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of IL-6 found in tissue of the same type as the sample but from an individual lacking breast cancer metastasis to bone. The Jagged1 high level expression marker may be a level of IL-6 in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of IL-6 found in tissue of the same type as the sample but from an individual having breast cancer but lacking breast cancer metastasis to bone. The Jagged1 high level expression marker may be a level of IL-6 in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of IL-6 found in a control sample.

The Jagged1 high level expression marker may be a level of IL-6 mRNA in the sample that is higher than the level of IL-6 mRNA found in normal tissue of the same type as the sample. The Jagged1 high level expression marker may be a level of IL-6 mRNA in the sample that is higher than the level of IL-6 mRNA found in tissue of the same type as the sample but from an individual lacking breast cancer metastasis to bone. The Jagged1 high level expression marker may be a level of IL-6 mRNA in the sample that is higher than the level of IL-6 mRNA found in tissue of the same type as the sample but from an individual having breast cancer but lacking breast cancer metastasis to bone. The Jagged1 high level expression marker may be a level of IL-6 mRNA in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of IL-6 mRNA found in normal tissue of the same type as the sample. The Jagged1 high level expression marker may be a level of IL-6 mRNA in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of IL-6 mRNA found in tissue of the same type as the sample but from an individual lacking breast cancer metastasis to bone. The Jagged1 high level expression marker may be a level of IL-6 mRNA in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of IL-6 mRNA found in tissue of the same type as the sample but from an individual having breast cancer but lacking breast cancer metastasis to bone. The Jagged1 high level expression marker may be a level of IL-6 mRNA in the sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the level of IL-6 mRNA found in a control sample.

The Jagged1 high level expression marker may be a level of IL-6R, IL-6R downstream signal transducers, IL-6R mRNA, or IL-6R downstream signal transducer mRNA that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 higher than the respective amount of IL-6R, IL-6R downstream signal transducers, IL-6R mRNA, or IL-6R downstream signal transducer mRNA in a control sample.

The method for diagnosing may also include diagnosing the subject as having an increased risk of breast cancer bone metastasis upon determining the presence of the Jagged1 high level expression marker in the sample. The method may also include diagnosing the subject as having decreased sensitivity to RANK or RANKL targeting treatments upon determining the presence of the Jagged1 high level expression marker in the sample. The RANK or RANKL targeting treatment at issue may be treatment with a monoclonal antibody targeting RANK or RANKL. The monoclonal antibody may be denosomab. The method may also include diagnosing the subject as having increased sensitivity to at least one of NOTCH targeting treatments or Jagged1 targeting treatments upon determining the presence of the Jagged1 high level expression marker in the sample. The NOTCH or Jagged1 targeting treatments at issue may include administering any one or more cancer treating drug herein, which include but are not limited to antibodies against the respective targets, GSIs, MRK-003, or antisense RNAs.

The step of obtaining may include harvesting the sample from the subject. Harvesting the sample from the subject may include at least one of a breast tissue biopsy, a breast cancer tumor biopsy, obtaining serum, obtaining a bone aspirate, obtaining a bone marrow biopsy, circulating tumor cell, or a metastatic tumor biopsy. The sample may be a serum sample, a breast tissue sample, a breast cancer tumor sample, a bone sample, a bone marrow aspirate, a bone marrow sample, a circulating tumor cell, or a metastatic tumor from the subject.

In an embodiment, the step of obtaining in the method for diagnosing is receiving the harvested sample from a party. The party may be the individual or entity that harvested the sample or an intermediate person or intermediate entity that first received the sample from either 1) the individual or entity that harvested the sample, or 2) a prior individual or prior entity that received the sample anywhere in the chain between the subject to the agent receiving the harvested sample.

The step of obtaining may include both harvesting the sample from the subject, and receiving the harvested sample from a party. The party may be the individual that harvested the sample or an intermediate person or intermediate entity. The intermediate person or intermediate entity may be a party that first received the sample from either another intermediate, or the individual that harvested the sample.

The method for diagnosing may also include obtaining a control sample. The control sample may be a serum sample control, normal tissue, normal breast tissue, normal bone tissue, non-tumor breast tissue, non-metastatic breast tumor tissue, normal serum, or a serum sample from an individual lacking breast cancer bone metastasis. The Jagged1 high level expression marker may be the presence of a Jagged1, Jagged1 mRNA, IL-6, or IL-6 mRNA in a sample that is at least 0.5 fold, 0.6 fold, 0.7 fold, 0.8 fold, 0.9 fold, 1.0 fold, 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, or 3.0 fold higher than the respective level of Jagged1, Jagged1 mRNA, IL-6, or IL-6 mRNA in one of these control samples.

The subject in a method for diagnosing herein may be a patient. The subject may be a breast cancer patient. The patient may be human or a non-human animal. Preferably, the patient is human.

The determining step in the method for diagnosing may include detecting the amount of Jagged1 in the sample, detecting the amount of Jagged1 in the control sample, and comparing the amount of Jagged1 in the sample to the amount of Jagged1 in the control sample. The detecting includes analysis of the sample and the control sample with a composition including an anti-Jagged1 antibody. Detecting may include an immunohistochemical analysis of the sample and the control sample with a composition including an anti-Jagged1 antibody. Any method of detecting Jagged1 known in the art or described by the embodiments or examples herein may be implemented to detect Jagged1 in the method for diagnosing. In an embodiment, the sample and control samples utilized for the determining step are a breast tumor sample from the subject and a non-tumor breast tissue sample, respectively. In an embodiment, the sample and control samples utilized for the determining step are a serum sample from the subject and a serum sample from an individual lacking breast cancer bone metastasis, respectively.

The determining step may be detecting the amount of Jagged1 mRNA in the sample and the amount of Jagged1 mRNA in the control sample. In an embodiment, the sample and control samples utilized for the determining step are a breast tumor sample from the subject and a non-tumor breast tissue sample, respectively.

Detecting Jagged1 or Jagged1 mRNA may be accomplished by any method known in the art or described in an embodiment or example herein. Jagged1 or Jagged1 mRNA may be detected by assaying DNA, RNA, SAGE, RNA-Seq., qRT-PCR, western analysis, IHC, FACS, or ELISA.

The determining step may be detecting the amount of IL-6 in the sample, detecting the amount of IL-6 in the control sample, and comparing the amount of IL-6 in the sample to the amount of IL-6 in the control sample. In an embodiment, the an amount of IL-6 in the sample that is at least 2-fold greater than the amount of IL-6 in the control sample is the Jagged1 high level expression marker.

Detecting IL-6 or IL-6 mRNA may be accomplished by any method known in the art or described in an embodiment or example herein. IL-6 or IL-6 mRNA may be detected by assaying DNA, RNA, SAGE, RNA-Seq., qRT-PCR, western analysis, IHC, or ELISA.

Detecting IL-6 may include ELISA with a composition including an anti-IL-6 antibody. In embodiment, the sample is at least one of a serum sample or a bone aspirate from the subject when IL-6 is to be detected, and the control is a serum control sample from an individual lacking breast cancer bone metastasis or a bone aspirate from an individual lacking breast cancer bone metastasis.

Detecting may include contacting anti-IL-6 antibody to bone aspirates, IL-6 staining of bone marrow, or staining of IL-6 downstream pathway moieties in metastatic tumors; the respective samples for such a detecting step are bone aspirates from the subject having breast cancer, bone marrow from the subject having breast cancer, metastatic tumors from the subject having breast cancer; and the respective control samples for such a detecting step are bone aspirates from non-metastatic bone, bone marrow from non-metastatic bone, non-tumor breast tissue.

An embodiment includes a method of treating a breast cancer patient. The method includes administering to the breast cancer patient at least one therapy selected from the group consisting of Notch targeting treatments and Jagged1 targeting treatments. The step of administering occurs after a determination of the presence of a Jagged1 high level expression marker in a sample from the breast cancer patient. The method of treating may include determination of the presence of a Jagged1 high level expression marker in a sample from the patient performed by any one of the methods of diagnosis herein.

An embodiment includes a method of treating a breast cancer patient including determining the presence of a Jagged1 high level expression marker in a sample from the patient performed by any one of the methods of diagnosis herein followed by administering to the breast cancer patient at least one therapy selected from the group consisting of Notch targeting treatments and Jagged1 targeting treatments. The step of administering occurs after a determination of the presence of a Jagged1 high level expression marker in a sample from the breast cancer patient.

The therapy in the method of treating may include administering an agent selected from any cancer treating drug targeting breast cancer bone metastasis. The therapy in the method of treating may include administering at least one agent selected from the group consisting of a Jagged1 activity down regulator, a Jagged1 gene expression down regulator, and an RNAi molecule that has a nucleotide sequence complementary to at least a portion of Jagged1 mRNA. The agent may be an shRNA as the RNAi molecule or DNA encoding the same, where the shRNA includes a nucleotide sequence having at least 70, 75, 80, 85, 90, 91, 92, 93, 94, 95, 96, 97, 98, 99, or 100% identity to a reference sequence consisting of the RNA sequence corresponding to one of SEQ ID NO: 74, SEQ ID NO: 77, SEQ ID NO: 80, SEQ ID NO: 83, SEQ ID NO: 86, SEQ ID NO: 89., SEQ ID NO: 92, SEQ ID NO: 95, SEQ ID NO: 98 and SEQ ID NO: 101. The percent identity may be 100% identity. The shRNA may include sequences as represented in one of SEQ ID NOs: 74, 77, 80, 83, 86, 89, 92, 95, 98 and 101 or include fragments thereof. One type of fragments that may be provided in an shRNA construct are the sense and antisense fragments specific for Jagged1 mRNA. The sense and antisense fragments for SEQ ID NO: 74 are AAGGTGTGTGGGGCCTCGGGT [SEQ ID NO: 72] and ACCCGAGGCCCCACACACCTT [SEQ ID NO: 73], respectively. The sense and antisense fragments for SEQ ID NO: 77 are CCTTTAACAAGGAGATGAT [SEQ ID NO: 75] and ATCATCTCCT TGTTAAAGG [SEQ ID NO: 76], respectively. The sense and antisense fragments for SEQ ID NO: 80 are CGTACAAGTAGTTCTGTAT [SEQ ID NO: 78] and ATACAGAACTACTTGTACG [SEQ ID NO: 79], respectively. The sense and antisense fragments for SEQ ID NO: 83 are CCCAGAATACTGATGGAAT [SEQ ID NO: 81] and ATTCCATCAGTATTCTGGG [SEQ ID NO: 82], respectively. The sense fragments for SEQ ID NO: 86 are GCTAGTTGAATACTTGAAT [SEQ ID NO: 84] and GCTAGTTGAATACTTGAAC [SEQ ID NO: 102]. The antisense fragments for SEQ ID NO: 86 are GTTCAAGTATTCAACTAGC [SEQ ID NO: 85] and ATTCAAGTATTCAACTAGC [SEQ ID NO: 103]. The sense and antisense fragments for SEQ ID NO: 89 are CCAGTAAGATCACTGTTTA [SEQ ID NO: 87] and TAAACAGTGATCTTACTGG [SEQ ID NO: 88], respectively. The sense and antisense fragments for SEQ ID NO: 92 are GGAGTATTCTCATAAGCTA [SEQ ID NO: 90] and TAGCTTATGAGAATACTCC [SEQ ID NO: 91], respectively. The sense fragments for SEQ ID NO: 95 are GCTAGTTGAATACTTGAAT [SEQ ID NO: 93], and GCTAGTTGAATACTTGAAC [SEQ ID NO: 102]. The antisense fragments for SEQ ID NO: 95 are GTTCAAGTATTCAACTAGC [SEQ ID NO: 94], ATTCAAGTATTCAACTAGC [SEQ ID NO: 103]. The sense and antisense fragments for SEQ ID NO: 98 are CCAGTTAGATCACTGTTTA [SEQ ID NO: 96] and TAAACAGTGATCTAACTGG [SEQ ID NO: 97], respectively. The sense and antisense fragments for SEQ ID NO: 101 are GGAACAGACTGAGCTATAT [SEQ ID NO: 99] and ATATAGCTCAGTCTGTTCC [SEQ ID NO: 100], respectively. Embodiments of the method of treating include shRNA utilizing one or more sets of sense and antisense fragments having have at least 70, 75, 80, 85, 90, 91, 92, 93, 94, 95, 96, 97, 98, 99, or 100% identity to reference sequences consisting of the RNA sequence corresponding to one of the sets selected from SEQ ID NO: 72 and SEQ ID NO: 73; SEQ ID NO: 75 and SEQ ID NO: 76; SEQ ID NO: 78 and SEQ ID NO: 79; SEQ ID NO: 81 and SEQ ID NO: 82; SEQ ID NO: 84, SEQ ID NO: 85, SEQ ID NO: 102, and SEQ ID NO: 103; SEQ ID NO: 87 and SEQ ID NO: 88; SEQ ID NO: 90 and SEQ ID NO: 91; SEQ ID NO: 93, SEQ ID NO: 94, SEQ ID NO: 102 and SEQ ID NO: 103; SEQ ID NO: 96 and SEQ ID NO: 97; SEQ ID NO: 99 and 100; SEQ ID NO: 104 and SEQ ID NO: 105; and SEQ ID NO: 106 and SEQ ID NO: 107. The sets of sense and antisense fragments may be joined by appropriate spacer sequences. Spacer sequences are exemplified, but not limited, by reference to SEQ ID NOS: 74, 77, 80, 83, 86, 89, 92, 95, 98, and 101. The shRNA may have a nucleotide sequence complementary to at least a portion of Jagged1 mRNA, and the DNA encoding the shRNA molecule may have a nucleotide sequence complementary to the corresponding portion of Jagged1 mRNA. The agent may be combined with a pharmaceutically acceptable carrier. Administering the RNAi molecule may be accomplished by any means known in the art, including administering a DNA encoding the RNAi molecule, a vector encoding the RNAi molecule, a recombinant virus encoding the RNAi molecule, an RNAi molecule with modified nucleotides, or an DNA encoding the RNAi molecule with modified nucleotides. Methods, compounds, modifications, and delivery schemes for administering the RNAi molecule that could be employed are described in Zhang, Y. and Huang, L. (2011) RNA Drug Delivery Approaches, in Drug Delivery in Oncology: From Basic Research to Cancer Therapy (eds F. Kratz, P. Senter and H. Steinhagen), Wiley-VCH Verlag GmbH & Co. KGaA, Weinheim, Germany. doi: 10.1002/9783527634057. ch 42, which is incorporated herein by reference as if fully set forth.

An embodiment includes a composition comprising at least one agent selected from the group consisting of a Jagged1 activity down regulator, a Jagged1 gene expression down regulator, an RNAi molecule that has a nucleotide sequence complementary to at least a portion of Jagged1 mRNA, and a DNA encoding the RNAi molecule that has a nucleotide sequence complementary to at least a portion of Jagged1 mRNA. The RNAi molecule may be an shRNA having a nucleotide sequence having at least, 75, 80, 85, 90, 91, 92, 93, 94, 95, 96, 97, 98, 99, or 100% identity to a reference sequence consisting of the RNA sequence corresponding to one of SEQ ID NO: 74, SEQ ID NO: 77, SEQ ID NO: 80, SEQ ID NO: 83, SEQ ID NO: 86, SEQ ID NO: 89., SEQ ID NO: 92, SEQ ID NO: 95, SEQ ID NO: 98 and SEQ ID NO: 101. The percent identity may be 100%. The shRNA in an embodiment of the composition may have one or more of the sets of sense and antisense fragments having at least 70, 75, 80, 85, 90, 91, 92, 93, 94, 95, 96, 97, 98, 99, or 100% identity to reference sequences consisting of the RNA sequence corresponding to one of the sets selected from SEQ ID NO: 72 and SEQ ID NO: 73; SEQ ID NO: 75 and SEQ ID NO: 76; SEQ ID NO: 78 and SEQ ID NO: 79; SEQ ID NO: 81 and SEQ ID NO: 82; SEQ ID NO: 84, SEQ ID NO: 85, SEQ ID NO: 102 and SEQ ID NO: 103; SEQ ID NO: 87 and SEQ ID NO: 88; SEQ ID NO: 90 and SEQ ID NO: 91; SEQ ID NO: 93, SEQ ID NO: 94, SEQ ID NO: 102 and SEQ ID NO: 103; SEQ ID NO: 96 and SEQ ID NO: 97; SEQ ID NO: 99 and 100; SEQ ID NO: 104 and SEQ ID NO: 105; and SEQ ID NO: 106 and SEQ ID NO: 107. The sets of sense and antisense fragments may be joined by appropriate spacer sequences. Spacer sequences are exemplified, but not limited, by reference to SEQ ID NOS: 74, 77, 80, 83, 86, 89, 92, 95, 98, and 101. The shRNA may have a nucleotide sequence complementary to at least a portion of Jagged1 mRNA, and the DNA encoding the shRNA molecule may have a nucleotide sequence complementary to the corresponding portion of Jagged1 mRNA. The composition may also include a pharmaceutically acceptable carrier.

As used herein, a pharmaceutically acceptable carrier may be any known to the skilled artisan. A pharmaceutically acceptable carrier may include at least one substance selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.) and phosphate buffered saline (PBS).

An embodiment includes a second method of treating a breast cancer patient. The second method includes administering to the breast cancer patient at least one second therapy selected from the group consisting of RANK targeting treatments and RANKL targeting treatments. The second therapy may be administering to the breast cancer patient denosumab. The step of administering may occur after a determination of the absence of a Jagged1 high level expression marker in a sample from the breast cancer patient. The method of treating may include determination of the absence of a Jagged1 high level expression marker in a sample from the patient performed by any one of the methods of diagnosis herein.

An embodiment includes a second method of treating a breast cancer patient including determining the absence of a Jagged1 high level expression marker in a sample from the patient performed by any one of the methods of diagnosis herein followed by administering to the breast cancer patient at least one therapy selected from the group consisting of RANK targeting treatments and RANKL targeting treatments. The step of administering occurs after a determination of the absence of a Jagged1 high level expression marker in a sample from the breast cancer patient.

Further embodiments herein may be formed by supplementing any single embodiment with one or more element from another embodiment, or replacing one or more element in any single embodiment with one or more element from another embodiment.

### EXAMPLES

The following non-limiting examples are provided to illustrate discoveries, particular embodiments or details therein. The embodiments throughout may be supplemented with one or more detail from any one or more example below. One or more element in embodiments throughout may be replaced by one or more detail below.

### Example 1

### The Notch Ligand Jagged1 is Associated with Breast Cancer Bone Metastasis

Expression profiling of human MDA-MB-231 (MDA231) breast cancer sublines with distinct bone metastatic abilities (Kang et al., 2003, which is incorporated herein by reference as if fully set forth) revealed that JAGGED1 (JAG1) levels were significantly elevated in aggressive bone-tropic sublines compared to the weakly metastatic ones (Sethi et al., 2011, which is incorporated herein by reference as if fully set forth). These findings suggested a link between tumor expression of Notch ligands and breast cancer bone metastasis.

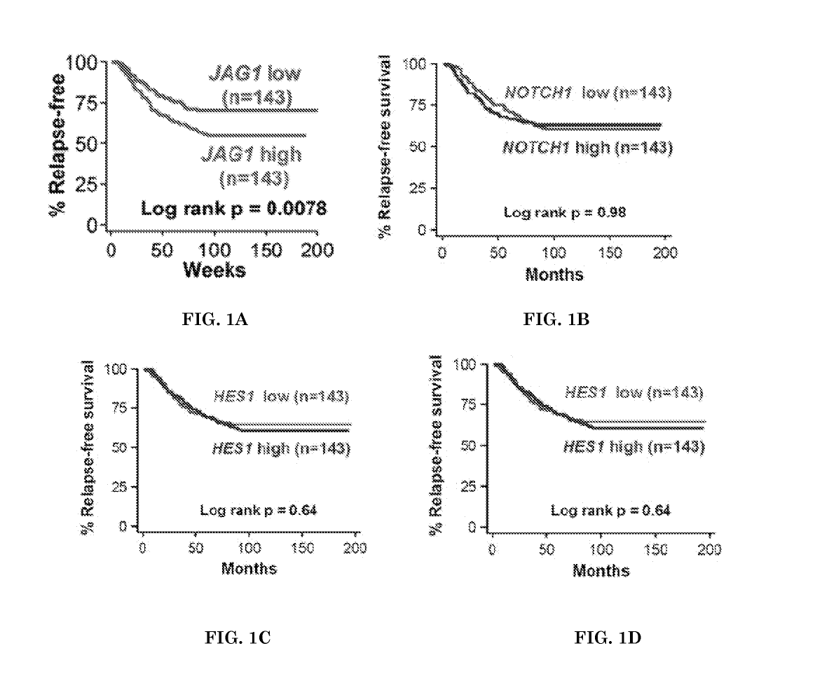

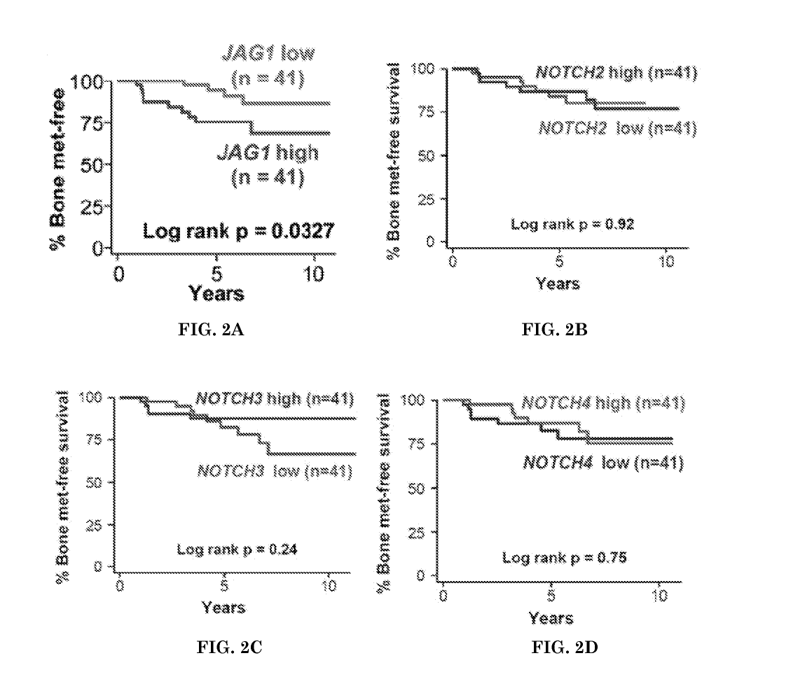

To determine the clinical significance of Jagged1 in breast cancer metastasis, its expression pattern was examined in tumor samples from patients in two previously reported data sets. The Wang data set (Wang et al., 2005, which is incorporated herein by reference as if fully set forth) revealed that JAG1 expression was significantly higher in patients with relapse (p=0.0045, Student's t test). Moreover, incidence of relapse was significantly greater in patients with high JAG1 expression compared to those with low expression (FIG. 1A). In contrast the incidence of relapse was not significantly different in patients with low or high expression of NOTCH1 or HES1 (FIGS. 1B-1D). Distinct from the Wang data set, the Minn data set (Minn et al., 2005, which is incorporated herein by reference as if fully set forth) includes more diverse clinical criteria such as organ-specific metastasis. The incidence of bone metastasis was significantly greater in patients with high JAG1 expression compared to those with low expression (FIG. 2A). In contrast the incidence of bone metastasis was not significantly different between patients with differential expression of NOTCH2, NOTCH3, and NOTCH4 (FIGS. 2B-2D) (NOTCH1 expression is too low for analysis). These findings further implicate Jagged1, in contrast to the Notch receptors or other pathway components, as a clinically significant player in breast cancer metastasis to the bone.

### Example 2

### Jagged1 Mediates Breast Cancer Bone Metastasis

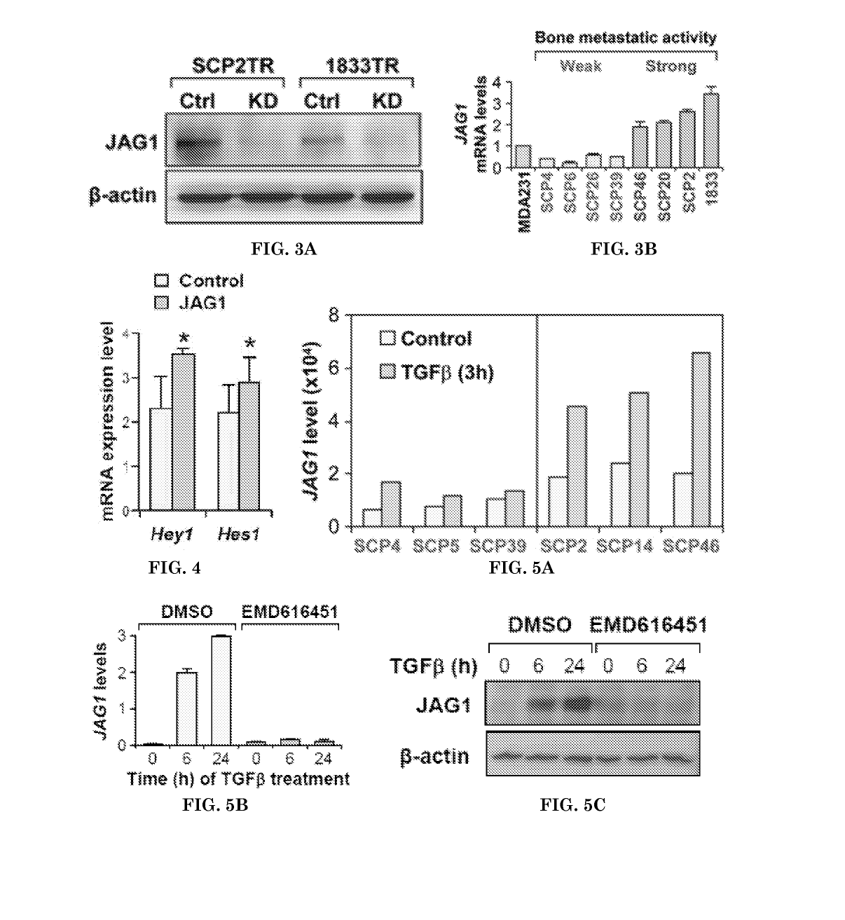

To directly test whether Jagged1 is functionally important for breast cancer bone metastasis, a short-hairpin RNA (shRNA) was used to stably silence JAG1 expression in SCP2 and 1833, which are two highly bone metastatic MDA231 sublines with high expression of JAG1. See FIGS. 3A and 3B. The progression of bone metastasis after intracardiac injection of tumor cells was monitored by weekly bioluminescence imaging (BLI) using a stably expressed firefly luciferase reporter. JAG1 knockdown (KD) significantly extended survival and delayed the onset of bone metastasis in mice. Despite no difference at early time points, BLI analysis showed that JAG1 KD reduced the bone tumor burden by 6- to 10-fold 3 weeks after injection, suggesting that tumor derived Jagged1 is necessary for efficient outgrowth of bone lesions. It was confirmed that the differences in BLI measurement of bone tumor burden corresponded to those achieved by histomorphometric and X-ray analyses.

Consistent with these results, histological analysis demonstrated a 2-fold decrease in the number of tartrate-resistant acid phosphatase-positive (TRAP+) osteoclasts along the bone tumor interface of bone lesions generated by JAG1 KD cells. Importantly, JAG1 KD did not alter the ability of tumor cells to proliferate in culture or as mammary tumors in mice. These results support a functional role for tumor-derived Jagged1 in bone metastasis, in part by its ability to support efficient tumor outgrowth and induce osteolysis.

Jagged1 was overexpressed in the mildly metastatic MDA231 subline SCP28 to determine whether enforced expression of Jagged1 is sufficient to promote bone metastasis. Mice injected with JAG1 overexpressing (OE) tumor cells had an earlier onset of bone metastasis, demonstrated a significant increase in bone metastasis burden by BLI, and developed severe osteolytic bone lesions as determined by X-ray and histological analysis. Ki67 staining of bone metastases revealed a greater number of proliferating cancer cells in the JAG1 OE group. In contrast, JAG1 OE did not increase the proliferation of tumor cells in culture or as primary mammary tumors, and did not affect their invasive ability in vitro. Importantly, it was found that Notch pathway target genes were elevated in the tumor-associated stroma of JAG1 OE bone metastases (FIG. 4) using mouse-specific RT-PCR analysis. These findings indicate that enforced expression of Jagged1 is sufficient to promote osteolytic bone metastasis, potentially by activating the Notch pathway in the supporting bone microenvironment.

Considering the importance of the immune system in bone homeostasis (Pacifici, 2010, which is incorporated herein by reference as if fully set forth) and the pathogenesis of bone metastasis (Xu et al., 2009, which is incorporated herein by reference as if fully set forth), the analysis was extended to an immunocompetent mouse model for bone metastasis. Using the BALB/c-derived TM40D-MB murine breast cancer cell line (Li et al., 2008, which is incorporated herein by reference as if fully set forth), mouse Jagged1 was overexpressed and its ability to promote metastasis in vivo was tested. The results showed a significant increase in bone metastasis ability for the Jag1 OE group in both immunocompetent BALB/c and athymic nude mice. These findings suggest that immune cells are unlikely to play a critical role in mediating the bone metastasis-promoting function of tumor-derived Jagged1.

### Example 3

### Jagged1 is Regulated by the TGFβ-SMAD Signaling Axis in Bone Metastasis

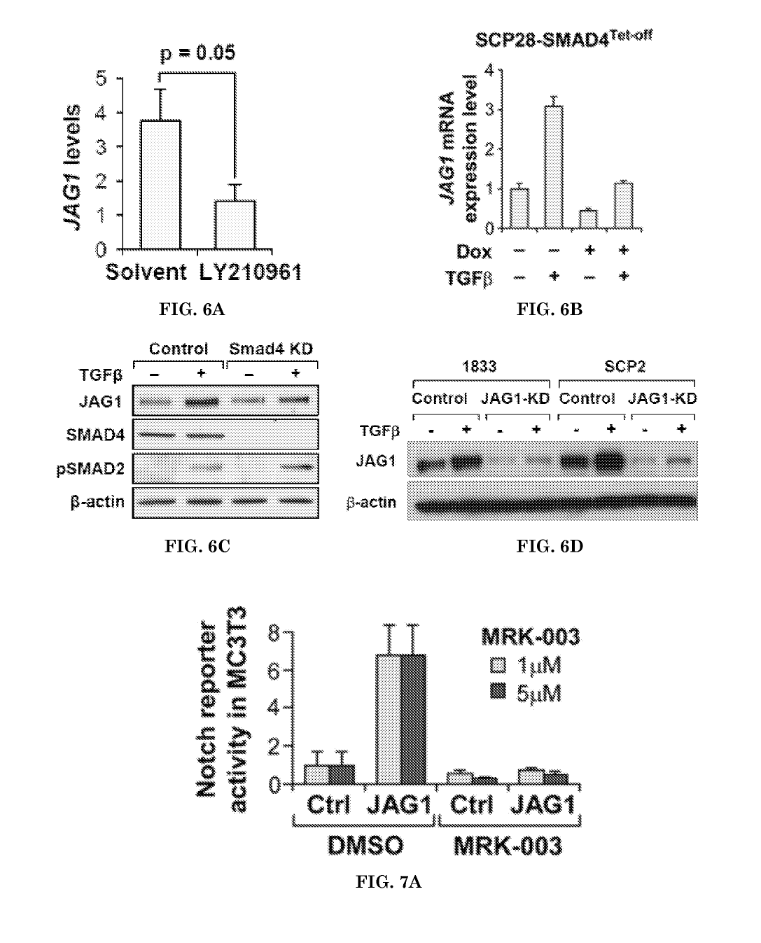

Expression of prometastatic genes is often influenced by signaling molecules present in the pathological milieu of the tumor microenvironment. To identify potential regulators of Jagged1 in the bone microenvironment, Enrichment of various signaling pathway target gene sets in the transcriptome of bone metastatic tumor cells was examined to identify potential regulators of Jagged1 in the bone microenvironment. Gene-set enrichment analysis demonstrated that TGFβ-responsive genes are significantly overrepresented among upregulated genes in bone metastatic MDA231 sublines. Notably, JAG1 was revealed among the 10-gene enrichment core of TGFβ responsive genes, suggesting that it is a potential target of TGFβ in breast cancer cells during osteolytic bone metastasis. Indeed, Jagged1 is potently upregulated in several breast cancer cell lines upon TGFβ stimulation (FIG. 5A). TGFβ Receptor 1 kinase inhibitor treatment abolished this induction in breast cancer cells in vitro (FIGS. 5B and 5C) and in bone metastases in vivo (FIG. 6A). Furthermore, using a previously reported SCP28 subline with conditional expression of SMAD4 (Korpal et al., 2009, which is incorporated herein by reference as if fully set forth), it was demonstrated a SMAD-dependent transcriptional regulation of JAG1 by TGFβ signaling (FIG. 6B; FIG. 6C).

It was investigated whether Jagged1 is an important downstream effector of the prometastatic TGFβ-SMAD signaling pathway during bone metastasis in vivo. As previously reported, SMAD4 KD significantly inhibits the development of osteolytic bone metastasis (Kang et al., 2005, which is incorporated herein by reference as if fully set forth). It was reasoned that if Jagged1 is an important TGFβ target during bone metastasis, overexpressing JAG1 in SMAD4 KD cells may partially restore their aggressive bone metastatic ability. Indeed, JAG1 OE strongly rescued the ability of SMAD4 KD tumor cells to generate osteolytic bone metastases. Furthermore, the reduced bone metastasis burden observed in the JAG1 KD experiments could also be explained in part by the inability of the JAG1 KD tumor cells to induce JAGGED1 expression in response to bone-derived TGFβ (FIG. 6D). Taken together, these findings demonstrate that TGFβ, a well-known prometastatic cytokine, stimulates Jagged1 expression in cancer cells to promote osteolytic bone metastasis.

### Example 4

### Jagged1 Confers a Growth Advantage by Activating Notch Signaling in Osteoblasts

Because manipulating Jagged1 expression influenced the development of bone metastasis without affecting primary tumor functions, it is likely that Jagged1-Notch signaling facilitates communication between tumor cells and the bone microenvironment to promote metastasis.

Therefore, the involvement of supporting bone cells, particularly osteoblasts and osteoclasts, was investigated in Jagged1-mediated bone metastasis by employing an in vitro coculture system.

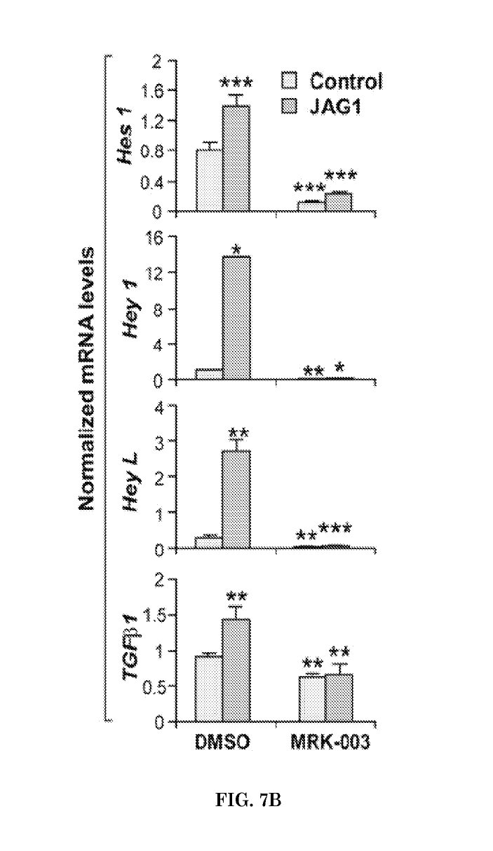

The ability of tumor-derived Jagged1 to activate the Notch pathway in associated osteoblasts was tested. When MC3T3-E1 osteoblasts expressing a Notch reporter (Zeng et al., 2005, which is incorporated herein by reference as if fully set forth) were cocultured with JAG1 OE tumor cells, a 6-fold increase in Notch activity was observed and the increase was abolished by the gamma-secretase inhibitor (GSI) MRK-003 (FIG. 7A). Moreover, osteoblasts separated by FACS from cocultured JAG1 OE GFP+ tumor cells demonstrated activation of several Notch target genes (Hes1, Hey1, HeyL and TGFβ1) that were downregulated by MRK-003 treatment (FIG. 7B).

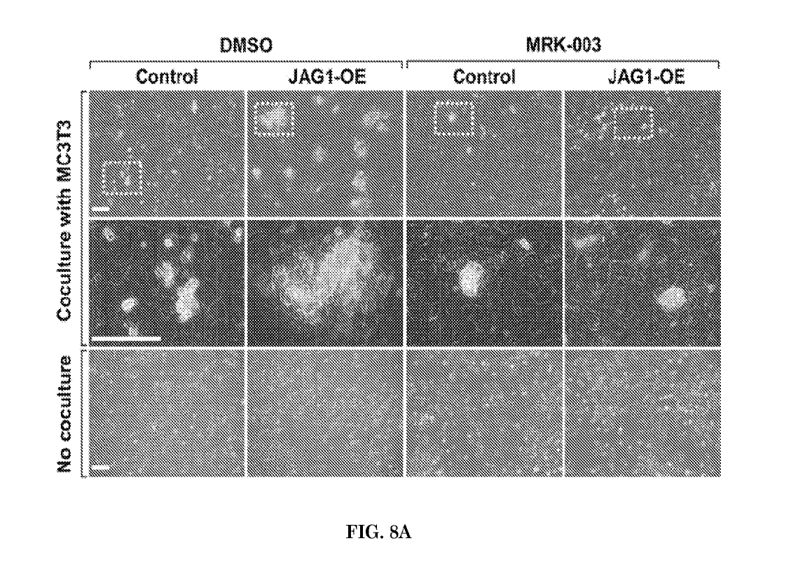

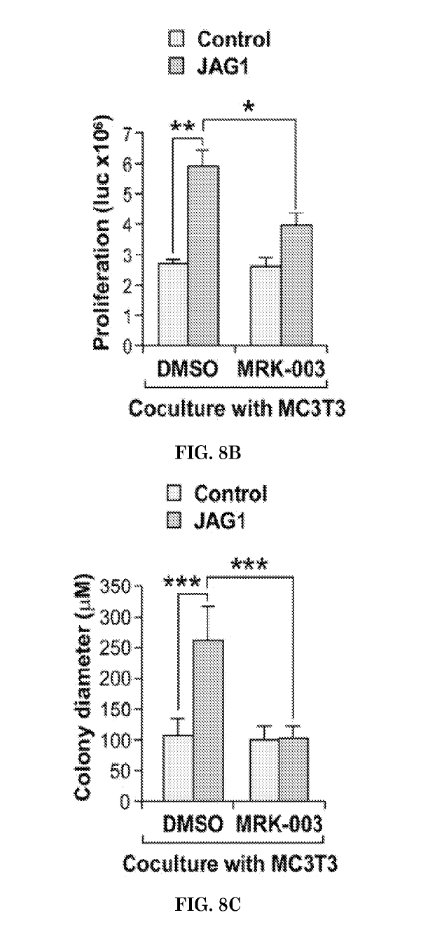

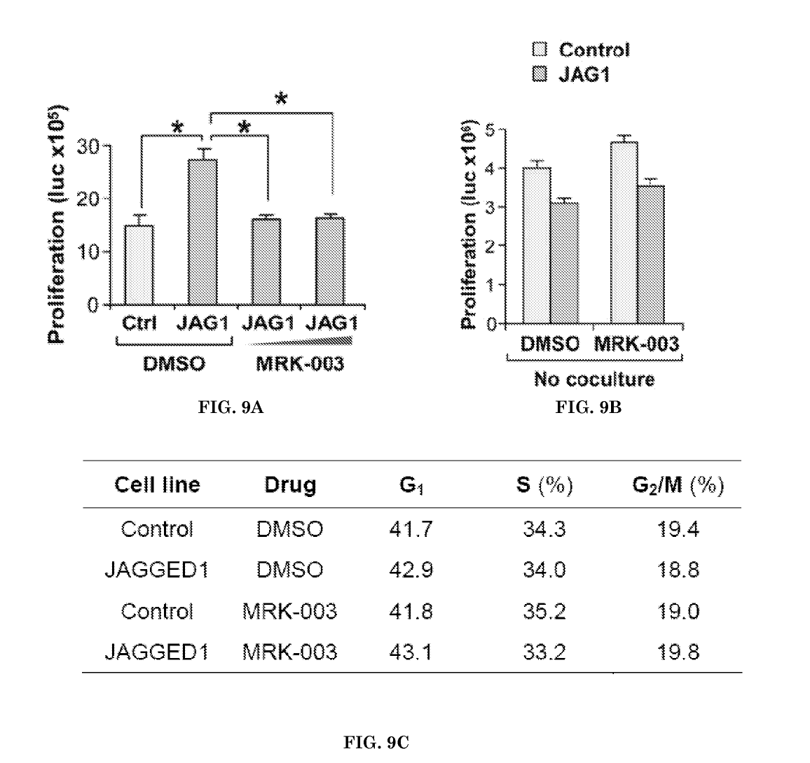

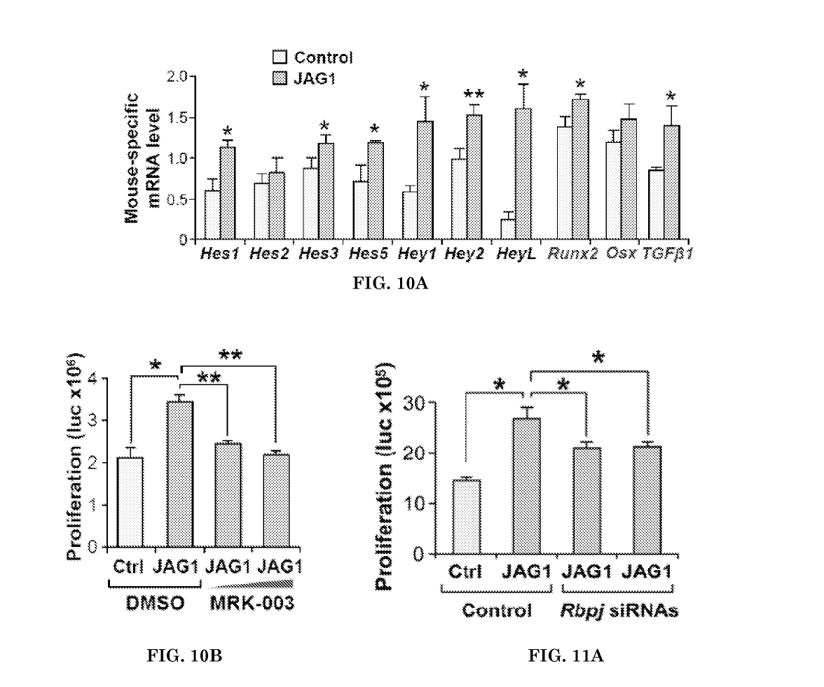

Considering the elevated proliferative index (Ki67+) of JAG1 OE bone metastases, it was investigated whether the growth advantage was acquired via interactions with osteoblasts. This was tested by culturing GFP⁺-luciferase labeled tumor cells over a monolayer of MC3T3-E1 osteoblasts and subsequently quantifying tumor proliferation via luciferase assay. The results showed a 2-fold increase in the number of JAG1 OE tumor cells compared to vector controls when normalized to the counts of either population cultured without osteoblasts (no coculture) (FIGS. 8A and 8B). Moreover, JAG1 OE tumor cells formed GFP+ colonies that were 2.5-fold larger in diameter (FIG. 8C). MRK-003 treatment abolished the growth advantage of JAG1 OE tumor cells in the osteoblast coculture (FIGS. 8A-8C and 9A) but did not affect their proliferative ability when cultured alone (FIGS. 9B-9C). These results were also confirmed in primary bone marrow osteoblast cocultures (FIGS. 10A and 10B). Furthermore, genetic inhibition of Notch signaling in MC3T3-E1 via siRNA-mediated silencing of Rbpj, an indispensable cofactor of the Notch pathway, diminished the ability of JAG1 to stimulate tumor cell proliferation in cocultures (FIG. 11A). Collectively, these findings revealed that activation of the Notch pathway in osteoblasts confers a proliferative advantage to JAG1 OE tumor cells.

To identify Jagged1-regulated genes in osteoblasts that are potentially required for the enhanced tumor growth properties, microarray profiling was performed on MC3T3-E1 cells that were FACS-separated from tumor cell cocultures. Transcriptomic profiling uncovered 123 genes that were activated by at least 3-fold in MC3T3-E1 cells cocultured with JAG1 OE tumor cells relative to controls. These genes were concomitantly downregulated in the MRK-003-treated groups (FIG. 11B). As expected, many well-characterized Notch targets were found among these candidate genes. The necessity of Hey1, the most upregulated downstream mediator of the Notch pathway, was investigated by silencing its expression in MC3T3-E1 (FIG. 12A). Hey1 KD in MC3T3-E1 significantly diminished the coculture growth of JAG1 OE tumor cells (FIG. 12B), suggesting that Hey1 is a required downstream mediator of Notch signaling in osteoblasts for promoting tumor growth.

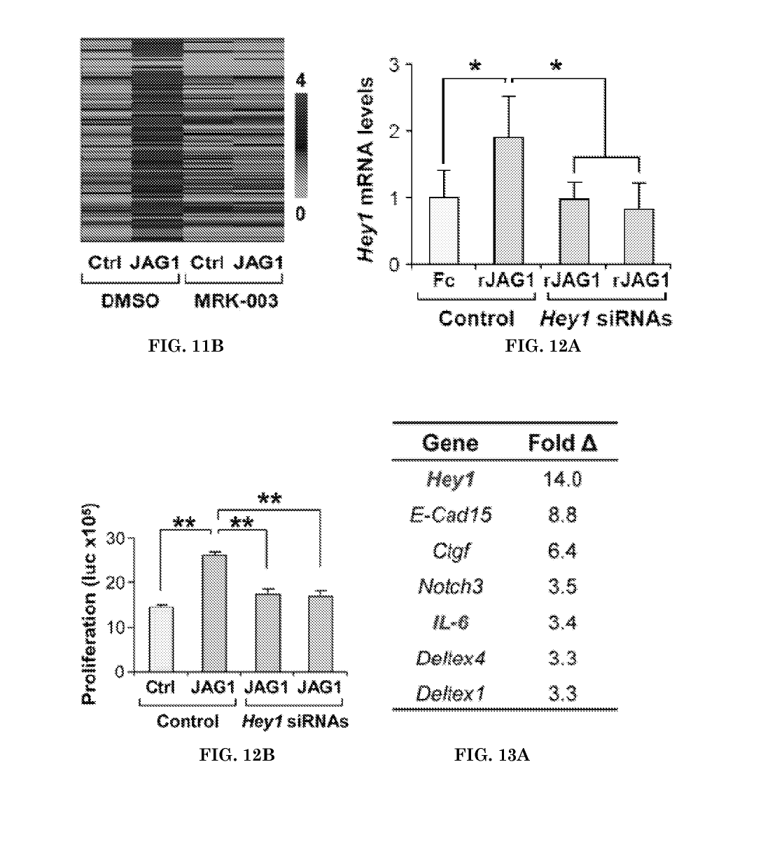

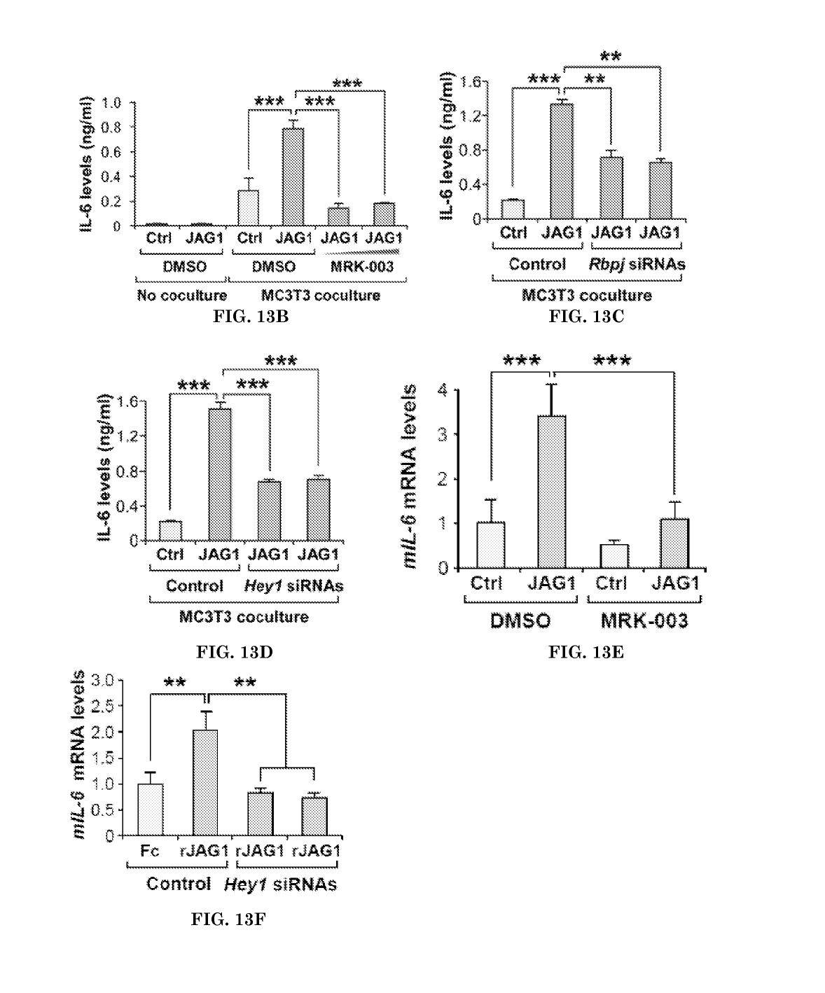

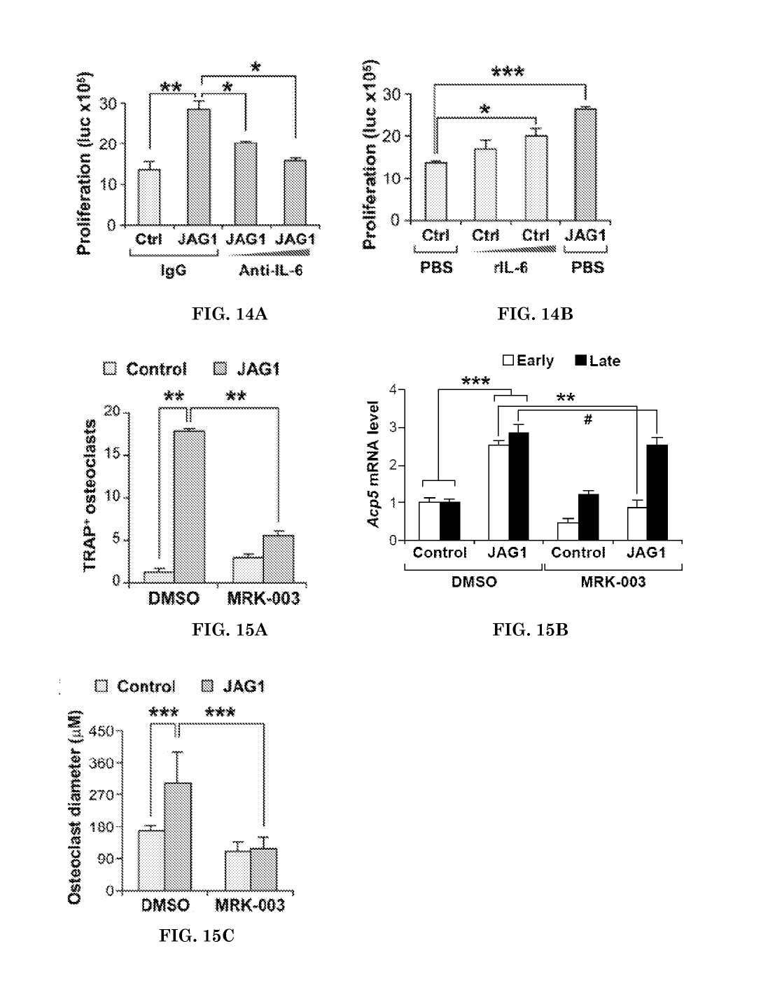

Next, Notch-dependent signaling proteins secreted by osteoblasts that may potentially stimulate tumor growth were identified. The most promising candidate from the ranked gene list was interleukin-6 (IL-6) (FIG. 13A) because it is implicated in the development of bone metastasis (Ara et al., 2009; de la Mata et al., 1995, which are incorporated herein by reference as if fully set forth) and associated with poor clinical outcome in patients with breast cancer (Salgado et al., 2003, which is incorporated by reference as if fully set forth). JAG1 OE cocultures demonstrated a 7-fold increase in IL-6 levels by ELISA (FIGS. 13B-13D). Importantly, IL-6 was selectively secreted by osteoblasts because conditioned media from tumor cells cultured alone contained negligible amounts of IL-6 (FIG. 13B); this is consistent with the observation that JAG1 OE promotes tumor cell growth only in the presence of MC3T3-E1 cells. IL-6 transcription and secretion from osteoblasts was dependent on the Notch pathway, as shown by MRK-003 and Rbpj siRNA treatments (FIGS. 13B and 13C; FIG. 13E). Furthermore, it was validated that Hey1 regulates both mRNA and protein levels of IL-6 (FIG. 13D; FIG. 13F). Based on these results, tests were conducted to analyze whether Notch-stimulated IL-6 secretion from osteoblasts was required for the enhanced tumor proliferation. Inhibition of osteoblast-derived IL-6 by a neutralizing antibody diminished the growth advantage of JAG1 OE tumor cells (FIG. 14A). Conversely, stimulation of control tumor cells by rIL-6 significantly enhanced their proliferative ability (FIG. 14B). These findings outline a positive feedback signaling axis by which Jagged1-Notch signaling stimulates the release of IL-6 from osteoblasts to promote tumor proliferation.

The important contribution of bone-derived TGFβ during osteolytic bone metastasis is well established. Bone is a rich reservoir of TGFβ, which is released into the bone microenvironment during osteolytic bone metastasis. Genetic or pharmacological disruption of TGFβ signaling potently reduces the development of bone metastasis, supporting the importance of the TGFβ pathway in supporting the bone metastatic ability of tumor cells (Korpal et al., 2009; Yin et al., 1999, which are incorporated herein by reference as if fully set forth). However, the functional downstream targets of the TGFβ-SMAD pathway in bone metastasis remain poorly defined. Here, it was shown that Jagged1 is a SMAD-dependent target of TGFβ in breast cancer bone metastasis and that reestablishing JAGGED1 expression in a SMAD4 KD background restores the potency of tumor cells to generate osteolytic bone metastasis. Thus, Jagged1 may mediate a positive feedback in response to bone-derived TGFβ during the vicious cycle of osteolytic bone metastasis. Intriguingly, an upregulation of the Tgfβ1 transcript in osteoblasts and osteoclasts upon activation of the Notch pathway was also observed (FIG. 7B). However, administration of a neutralizing antibody preventing the feedback of TGFβ on JAG1 OE tumor cells in osteoblast cocultures did not significantly alter their growth properties. Collectively, these studies suggest that the release of bone-derived TGFβ in response to osteolysis, as opposed to de novo expression of osteoblast derived TGFβ in response to Notch activation, is likely to be more critical in the pathogenesis of Jagged1-mediated bone metastasis. The Notch and TGFβ-signaling pathways have been shown to converge in diverse contexts such as epithelial to mesenchymal transition (Zavadil et al., 2004, which is incorporated herein by reference as if fully set forth) and the pathogenesis of glomerular disease (Niranjan et al., 2008, which is incorporated herein by reference as if fully set forth).

The results herein show that these two pathways once again link up to constitute a potent positive feedback loop between tumor cells and the bone microenvironment to promote osteolytic bone metastasis. Jagged1 was found to be a central mediator of Notch-TGFβ signaling crosstalk in bone metastasis.

An important stroma-dependent mechanism for the Notch ligand Jagged1 in promoting breast cancer metastasis to the bone is revealed herein. These studies also revealed the convergence of two developmentally conserved signaling pathways—TGFβ and Notch—in the pathological crosstalk between tumor cells, bone-specific cells, and the bone matrix during breast cancer bone metastasis. Robust evidence for GSIs as therapeutic agents against bone metastasis by targeting the tumor-associated stroma is provided.

### Example 5

### Tumor-Derived Jagged1 Directly Promotes Osteoclast Differentiation

The severe osteolytic phenotype observed in Jagged1-mediated bone metastases could be explained by two possible mechanisms. First, JAGGED1-expressing tumor cells may indirectly impact osteoclast activity by altering the expression of osteoblast derived Rankl and Opg. Second, and alternatively, JAG1 OE tumor cells may directly interact with pre-osteoclasts to stimulate their maturation. The first possibility was ruled out by the observation that there was no difference in mRNA and protein levels of Rankl and Opg in MC3T3-E1-tumor cell cocultures from each experimental condition. Moreover, the conditioned media from these cocultures did not impact osteoclast properties. Therefore, the second possibility was tested by directly coculturing tumor cells with pre-osteoclast Raw 264.7 cells. Strikingly, JAG1 OE cocultures showed a 15-fold increase in TRAP⁺ osteoclasts relative to controls, whereas MRK-003 treatment essentially abolished this phenotype (FIG. 15A). These findings were confirmed in primary osteoclast cocultures and by using recombinant JAGGED1 protein (rJAG1) alone, a different GSI (GSI IX), and an additional murine osteoclast precursor cell line (MOCP5). Delayed initiation of MRK-003 treatment (Late) failed to fully rescue the phenotype, as shown by AcpS (mouse gene encoding TRAP) mRNA levels (FIG. 15B), implying that JAGGED1 facilitates an early stage in osteoclast maturation. Furthermore, TRAP+ osteoclasts in JAG1 OE cocultures were significantly larger (FIG. 15C) and contained more nuclei, suggesting more efficient osteoclast fusion and accelerated differentiation. In contrast, cocultures treated with MRK-003 displayed smaller osteoclasts with fewer nuclei. To further validate these findings, profiles of mRNA expression levels of osteoclast differentiation markers in Raw 264.7 cells were developed. As anticipated, expression of several markers was elevated in the JAG1 OE cocultures and suppressed in the MRK-003-treated cocultures. Taken together, these results demonstrate that JAGGED1-expressing tumor cells are capable of directly activating osteoclasts and help provide a mechanistic explanation for the severe osteolytic phenotype observed in mice.

### Example 6

### Disruption of Notch Signaling in the Bone Microenvironment Reduces Bone Metastasis

GSIs may be utilized as a therapy against breast cancer bone metastasis. Disruption of the Notch pathway has been achieved through pharmacological inhibition of gamma-secretase, the enzymatic complex that mediates the final cleavage of the Notch receptor leading to release of its transcription-activating intracellular domain. These pharmacological agents, known as GSIs, are gaining recognition as potential anticancer agents (Rizzo et al., 2008, which is incorporated herein by reference as if fully set forth). However, it has not been definitively determined whether cancer progression is impeded by disrupting Notch signaling in the tumor cells or the associated stromal microenvironment. Moreover, a few studies have revealed a subset of cancer cell lines that are resistant to GSI treatment. Consistently, the proliferation assays and primary tumor xenografts of MDA231 sublines herein revealed no difference between control and MRK-003-treated groups, particularly at relatively low concentrations that were sufficient to inhibit the Notch pathway in bone-specific cells. These findings were supported by another study in which a panel of six breast cancer cell lines, including MDA231, were treated with three distinct GSIs, and no effect on proliferation/survival was observed for two of the compounds, whereas the third elicited cytostasis at concentrations similar to that of a proteosome inhibitor, suggesting nonspecific gamma-secretase-independent effects (Han et al., 2009, which is incorporated herein by reference as if fully set forth). An extensive series of experiments was used to show that MRK-003 disrupts bone-specific tumor functions by inhibiting the Jagged1-Notch mediated crosstalk between tumor cells and supporting bone cells. These findings support the application of GSIs as therapy against bone metastasis, most probably at a dosage that would circumvent drug-associated toxicities such as gastrointestinal irritation.

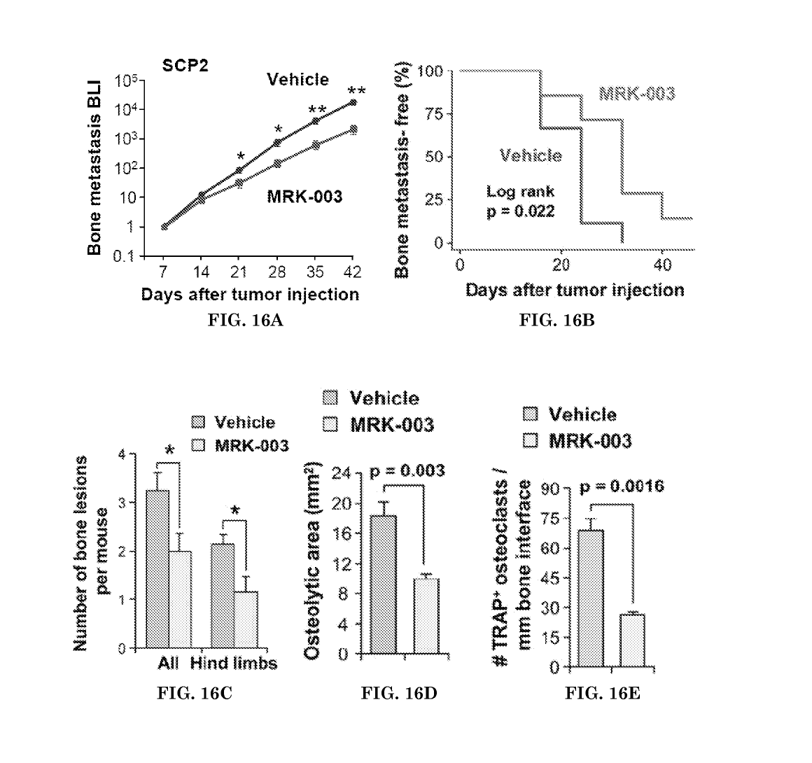

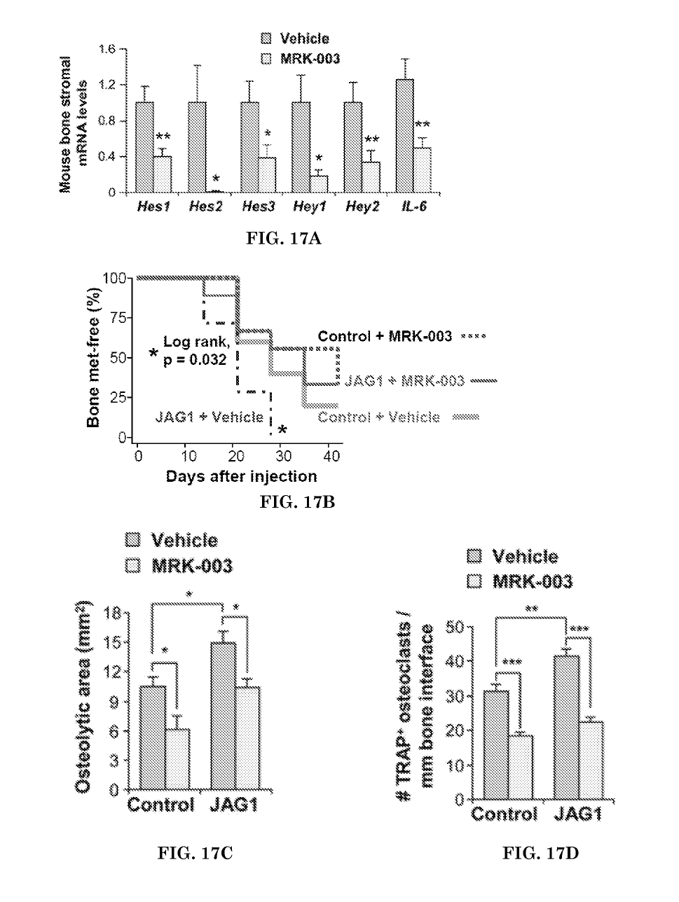

Tests were conducted to analyze whether MRK-003 treatment can reduce bone metastasis by targeting the supporting bone microenvironment. To this end, mice were inoculated with the aggressive bonetropic subline SCP2, which expresses high endogenous JAG1 levels, and concomitantly treated with MRK-003. MRK-003 treatment led to a 5-fold reduction in bone metastasis burden by BLI and an approximate 10-day delay in the onset of bone metastasis (FIGS. 16A-16B). The number of bone lesions was also reduced in the MRK-003-treated group (FIG. 16C), which was accompanied by a 2-fold reduction in X-ray lesion area (FIG. 16D) and a 3-fold decrease in the number of TRAP+ osteoclasts (FIG. 16E). In contrast the growth rate of primary mammary tumors was not altered by MRK-003 treatment, suggesting that direct targeting of Notch signaling in tumor cells cannot explain the reduced tumor burden in the bone metastasis experiments. It was also confirmed that MRK-003 treatment disrupted Notch signaling in the stromal compartment of bone metastases because expression levels of several Notch target genes, as well as IL-6, were significantly reduced in the stromal compartment of MRK-003-treated bone metastases, as measured by species-specific qRT-PCR (FIG. 17A). It was further tested whether MRK-003 treatment could reverse the severe bone metastasis phenotype induced by JAG1 OE. The significant increase in bone metastasis observed in the JAG1 OE group was reduced by more than 6-fold when the mice were treated with MRK-003, decreasing the tumor signal to levels found in the control group (FIGS. 17B-17C). Mirroring these changes in bone tumor dynamics, osteolysis was also reduced in MRK-003-treated mice (FIG. 17D). Overall, these studies confirm that the severe osteolytic bone metastasis phenotype mediated by Jagged1-expressing breast cancer is dependent on stromal Notch activation and is, therefore, susceptible to pharmacological inhibition of the Notch pathway in the bone microenvironment.

Elevated expression of Jagged1 in breast cancer cells promotes bone metastasis by activating the Notch pathway in supporting bone cells. Jagged1 is overexpressed in bone metastatic tumor cells and is further activated by the bone-derived cytokine TGFβ during osteolytic bone metastasis. Jagged1-expressing tumor cells acquire a growth advantage in the bone microenvironment by stimulating the release of IL-6 from osteoblasts and exacerbate osteolytic lesions by directly activating osteoclast maturation. GSI treatment reversed these prometastatic functions of Jagged1 by disrupting the Notch pathway in associated bone cells. The results herein support a distinct paradigm for the involvement of Notch signaling in the progression of breast cancer.

These investigations demonstrated that the Notch pathway receptors and select downstream targets are not associated with breast cancer progression. In contrast, it was unpredictably revealed that elevated expression of Notch pathway ligands is associated with metastatic ability of breast cancer cells. Furthermore, high expression of JAG1, in particular, was found to correlate with breast cancer bone metastasis in patient samples.

The coculture studies herein revealed that Jagged1 induces the expression and secretion of IL-6 from osteoblasts via activation of the Notch-signaling cascade, in turn conferring an osteoblast-dependent proliferative advantage to tumor cells. IL-6 is associated with a poor prognosis in breast cancer (Salgado et al., 2003, which is incorporated herein by reference as if fully set forth) and is capable of supporting tumor growth in the bone microenvironment (Sasser et al., 2007, which is incorporated herein by reference as if fully set forth). In neuroblastoma and multiple myeloma, stromal-derived IL-6 has been shown to be an important mediator between cancer cells and the bone microenvironment by supporting tumor survival and affecting osteoclast differentiation, respectively (Ara et al., 2009; Mitsiades et al., 2006, which are incorporated herein by reference as if fully set forth). In the present examples the pathological role of IL-6 is further extended to its involvement in Jagged1-mediated bone metastasis via an osteoblast-dependent positive feedback mechanism.

Overall, the in vivo and in vitro studies demonstrated a direct and strong impact of Jagged1 in promoting osteoclastogenesis and bone destruction.

### Example 7

### Summary

A new model in which the Notch pathway is activated in the tumor associated stromal microenvironment was discovered. It was discovered that the Notch pathway ligand Jagged1 is upregulated in breast cancer cells that have greater metastatic ability. It is also shown that Jagged1 expression is regulated by Smad-dependent signaling of the cytokine TGFβ, an important mediator of bone metastasis and a cytokine that is richly stored in bone matrix.

The Notch ligand Jagged1 was discerned to be a clinically and functionally important mediator of bone metastasis by activating the Notch pathway in bone cells. Jagged1 promotes tumor growth by stimulating IL-6 release from osteoblasts and directly activates osteoclast differentiation. Furthermore, Jagged1 is a potent downstream mediator of the bone metastasis cytokine TGFβ that is released during bone destruction. Importantly, gamma-secretase inhibitor treatment reduces Jagged1-mediated bone metastasis by disrupting the Notch pathway in stromal bone cells. These findings elucidate a stroma-dependent mechanism for Notch signaling in breast cancer and provide rationale for using gamma-secretase inhibitors for the treatment of bone metastasis.

Cell-lines were established that have been genetically manipulated to either overexpress Jagged1 using the pMSCV retroviral system or knocking down using the pRetroSuper retroviral system. An in vivo xenograft bone metastasis model was implemented by injecting these genetically manipulated human tumor cells into the left ventricle of mice allowing the tumor cells to enter circulation, disseminate throughout the body, and particularly colonize the bone. Preclinical treatment protocols included administering GSI to mice injected with tumor cells. The mice were treated with GSI twice a week at a concentration of 100 mg/kg. These in vivo studies led to the discovery that GSI inhibits bone metastasis by disrupting Notch signaling in the tumor stroma.

Using the in vivo mouse model, it was shown that functional knockdown and overexpression of Jagged1 in tumor cells leads to decreased and increased bone metastasis burden in mice, respectively. Moreover, it was demonstrated that Jagged1-expressing tumor cells activate the Notch pathway in tumor associated bone stromal cells, leading to increased tumor proliferation (Ki67 staining) and osteolytic lesions promoted by osteoclastogenesis (TRAP staining) in vivo.

In vitro functional analysis demonstrated that Jagged1-expressing tumor cells are directly responsible for the increased proliferation when co-cultured with osteoblasts and promote osteoclastogenesis by activating the Notch pathway in osteoclasts, both processes of which are susceptible to disrupting Notch signaling by gamma-secretase inhibitor (GSI) treatment or by genetic inhibition of Jagged1 by RNAi. Most importantly, mice injected with bone metastatic cell lines with high Jagged1 expression can be treated with Jagged1 or Notch targeting treatments, substantially decreasing bone metastasis compared to vehicle mice. Furthermore, Notch/Jagged1 targeting treatment rescued the bone metastatic phenotype of Jagged1-overexpressing cells by disrupting Notch signaling in the tumor stroma. These data collectively establish GSI as a novel therapeutic agent against breast cancer bone metastasis and establish a treatment model that targets the tumor microenvironment instead of the tumor itself.

Functional mechanisms that mediate tumor-stromal interactions through the Jagged1/Notch pathway were elucidated. Jagged1 overexpression in tumor cells stimulate the expression and productive of IL-6 from osteoblasts, which feed back to tumor cells to promote proliferation. Furthermore, Jagged1 directly promotes osteoclast differentiation and maturation through mechanisms that are independent of RANKL/RANK signaling. These results suggest that IL-6 targeting treatments, such as monoclonal antibodies against IL-6 or its receptor IL-6R, or small molecular inhibitors against the IL-6R downstream signal transducers, such as Jak2, can be used to treat bone metastasis induced by Jagged1. Furthermore, Jagged1 overexpression may render tumor cells insensitive to RANK targeting treatments (such as denosumab, monocloncal antibody against RANKL). Jagged1 (and potentially IL-6) can therefore serve as a tumor or serum marker to identify tumors that are likely to be refractory to denosumab treatments, but may respond to Jagged1 or Notch targeting therapies.

### Example 8

### shRNAs for RNAi

Jagged1 targeting treatments may include RNAi. shRNAs that may be used as agents for RNAi based Jagged1 targeting treatments are exemplified but not limited to the following.

hJagged1 shRNA #1: The DNA sequence corresponding to hJagged1 shRNA #1 is GATCTCCAAGGTGTGTGGGGCCTCGGGTTTCAA GAGAACCCGAGGCC CCACACACCTTTTTTTGGAAAAGCTTTTCCAAAAAAA GGTGTGTGGGGCCTCGG GTTCTCTTGAAACCCGAGGCCCCACACACCTTGG A [SEQ ID NO: 74], the sense strand is AAGGTGTGTGGGGCCTCGGGT [SEQ ID NO: 72] and the antisense strand is ACCCGAGGCCCCACACACCTT [SEQ ID NO: 73].

hJagged1 shRNA #2: The DNA sequence corresponding to hJagged1 shRNA #2 is GATCTCCCCTTTAACAAGGAGATGATTTCAAGAGAA TCATCTCCTT GTTAAAGGTTTTTGGAAAAGCTTTTCCAAAAACCTTTAACAA GGAGATGATTCTC TTGAAATCATCTCCTTGTTAAAGGGGA [SEQ ID NO: 77], the sense strand is CCTTTAACAA GGAGATGAT [SEQ ID NO: 75] and the antisense strand is ATCATCTCCT TGTTAAAGG [SEQ ID NO: 76].

hJagged1 shRNA #3: The DNA sequence corresponding to hJagged1 shRNA #3 is GATCTCCCGTACAAGTAGTTCTGTATTTCAAGAGAAT ACAGAACTACT TGTACGTTTTTGGAAAAGCTTTTCCAAAAACGTACAAGTA GTTCTGTATTCTCTT GAAATACAGAACTACTTGTACGGGA [SEQ ID NO: 80], the sense strand is CGTACAAGTA GTTCTGTAT [SEQ ID NO: 78] and the antisense strand is ATACAGAACT ACTTGTACG [SEQ ID NO: 79].

hJagged1 shRNA #4: The DNA sequence corresponding to hJagged1 shRNA #4 is GATCTCCCCCAGAATACTGATGGAATTTCAAGA GAATTCCATCAGTATTCTGGGTTTTTGGAAAAGCTTTTCCAAAAACCCAGA ATACTGATGGAATTCTCTT GAAATTC CATCAGTATTCTGGGGGA [SEQ ID NO: 83], the sense strand is CCCAGAATAC TGATGGAAT [SEQ ID NO: 81] and the antisense strand is ATTCCATCAG TATTCTGGG [SEQ ID NO: 82].

hJagged1 shRNA #5: The DNA sequence corresponding to hJagged1 shRNA #5 is GATCTCCGCTAGTTGAATACTTGAATTTCAAGA GAGTTCAAGTATT CAACTAGCTTTTTGGAAAAGCTTTTCCAAAAAGCTAGT TGAATACTTGAACTCTC TT GAAATTCAAGTATTCAACTAGCGGA [SEQ ID NO: 86], the sense strands are GCTAGTTGAATACTTGAAT [SEQ ID NO: 84] and GCTAGTTGAATACTTGAAC [SEQ ID NO: 102], and the antisense strands are GTTCAAGTATTCAACTAGC [SEQ ID NO: 85] and ATTCAAGTATTCA ACTAGC [SEQ ID NO: 103].

hJagged1 shRNA #6: The DNA sequence corresponding to hJagged1 shRNA #6 is GATCTCCCCAGTAAGATCACTGTTTATTCAAGAGA TAAACAGTGATCTTACTGGTTTTTGGAAAAGCTTTTCCAAAAACCAGTAAGAT CACTGTTTATCTCTT GAATAAACAGTGATCTTACTGGGGA [SEQ ID NO: 89], the sense strand CCAGTAAGAT CACTGTTTA [SEQ ID NO: 87] and the antisense strand is TAAACAGTGA TCTTACTGG [SEQ ID NO: 88].

mJagged1 shRNA #1: The DNA sequence corresponding to mJagged1 shRNA #1 is GATCTCCGGAGTATTCTCATAAGCTATTCAAGAGATA GCTTATGAGAATACTCCTTTTTGGAAAAGCTTTTCCAAAAAGGAGTATTCT CATAAGCTATCTCTT GAATAGCTTATGAGAATACTCCGGA [SEQ ID NO: 92], the sense strand is GGAGTATTCT CATAAGCTA [SEQ ID NO: 90] and the antisense strand is TAGCTTATGA GAATACTCC [SEQ ID NO: 91].

mJagged1 shRNA #2: The DNA sequence corresponding to mJagged1 shRNA #2 is GATCTCCGCTAGTTGAATACTTGAATTTCAAGAGAGT TCAAGTATTCAACTAGCTTTTTGGAAAAGCTTTTCCAAAAAGCTAGTTGAA TACTTGAACTCTCT TGAAATTCAAGTATTCAACTAGCGGA [SEQ ID NO: 95], the sense strands are GCTAGTTGAATACTTGAAT [SEQ ID NO: 93] and GCTAGTTGAATACTTGAAC [SEQ ID NO: 102], and the antisense strands are GTTCAAGTATTCAACTAGC [SEQ ID NO: 94] AND ATTCAAGTATTCAACTAG C [SEQ ID NO: 103].

mJagged1 shRNA #3: The DNA sequence corresponding to mJagged1 shRNA #3 is GATCTCCCCAGTTAGATCACTGTTTATTCAAGAGATA AACAGTGATCTAACTGGTTTTTGGAAAAGCTTTTCCAAAAACCAGTTAGAT CACTGTTTATCTCTT GAATAAACAGTGATCTAACTGGGGA [SEQ ID NO: 98], the sense strand is CCAGTTAGATCACTGTTTA [SEQ ID NO: 96] and the antisense strand is TAAACAGTGATCTAACTGG [SEQ ID NO: 97].

mJagged1 shRNA #4: The DNA sequence corresponding to mJagged1 shRNA #4 is GATCTCCGGAACAGACTGAGCTATATTTCAAGAGAAT ATAGCTCAGTCTGTTCCTTTTTGGAAAAGCTTTTCCAAAAAGGAACAGACT GAGCTATATTCTCT TGAAATATAGCTCAGTCTGTTCCGGA [SEQ ID NO: 101], the sense strand is GGAACAGACT GAGCTATAT [SEQ ID NO: 99] and the antisense strand is ATATAGCTCA GTCTGTTCC [SEQ ID NO: 100].

Additional mJagged1 strands: DNA sequences corresponding to additional mJagged1 strands include sense strand CCTTGATAGCATCACTTTA [SEQ ID NO: 104], antisense strand TAAAGTGATGCTATCAAGG [SEQ ID NO: 105]; and sense strand GCCTTAAGTGAGGAAATTA [SEQ ID NO: 106] and antisense strand TGATTTCCTCACTTAAGGC [SEQ ID NO: 107].

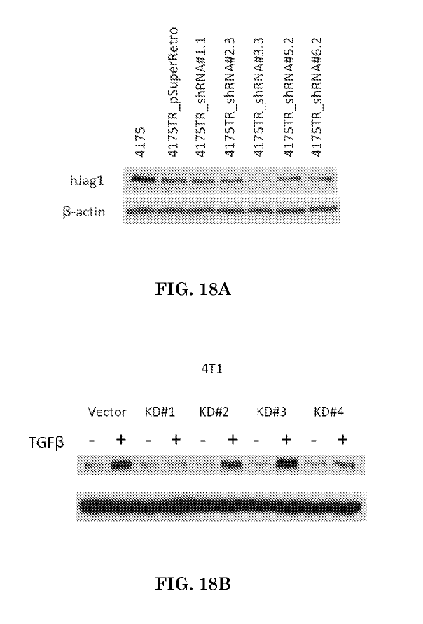

Western blots of Jagged1 knockdowns are illustrated in FIGS. 18A and 18B. FIG. 18A illustrates hJag1 protein levels in control and shRNA knockdown lines as follows: Lane 1, 4175 Jag1 expression control; lane 2, 4175TR_pSuperRetro vector control; lane 3, 4175TR_shRNA#1.1 [SEQ ID NO: 74]; lane 4, 4175TR_shRNA#2.3 [SEQ ID NO: 77]; lane 5, 4175TR_shRNA#3.3 [SEQ ID NO: 80]; lane 6, 4175TR_shRNA#5.2 [SEQ ID NO: 86]; lane 7, 4175TR_shRNA#6.2 [SEQ ID NO: 89]. FIG. 18B illustrates protein levels of Jagged1 in control and shRNA knockdown lines in response to a time course of TGFβ treatment as follows: lanes 1 and 2, vector control without and with TGFβ Treatment, lanes 3 and 4, KD#1 [SEQ ID NO: 92] without and with TGFβ Treatment, lanes 5 and 6, KD#2 [SEQ ID NO: 95] without and with TGFβ Treatment, lanes 7 and 8, KD#3 [SEQ ID NO: 98] without and with TGFβ Treatment, lanes 9 and 10, KD#4 [SEQ ID NO: 101] without and with TGFβ Treatment.

### Example 9

### Experimental Procedures

Tumor Xenografts and Bioluminescence Analysis

For bone metastsis studies, 10⁵ tumor cells were injected into the left cardiac ventricle of anesthetized female athymic Ncr-nu/nu or BALB/c mice. Development of metastases was monitored by BLI. Bioluminescence images were acquired with a Xenogen IVIS 200 Imaging System. Analysis was performed with Living Image software by measuring photon flux in the hindlimbs of mice. Data were normalized to the signal on day 7. Bone metastasis-free survival curves represent the time point at which each mouse developed bone metastasis by threshold BLI signals in the hindlimbs. For the orthotopic xenograft model, mammary fat pad injections and primary tumor size measurements were performed following the procedure described previously (Minn et al., 2005, which is incorporated herein by reference as if fully set forth).

Osteoblast Coculture, Gene Expression, and Microarray Analysis

MC3T3-E1 cells were seeded at 2×10⁵ cells/well in 12-well plates. After confluence was achieved, luciferase/GFP-labeled (GFP+) control and JAG1 OE cells were added at 1×10⁴ cells/well in triplicate and treated with DMSO or 1 μM MRK-003. Media supplemented with appropriate drugs were changed every 2 days. After 6 days the coculture was subjected to a luciferase assay to selectively quantify the number of tumor cells. These values were normalized against luciferase quantification of 12-well plates seeded with tumor cells alone.

For gene expression analysis, MC3T3-E1 cells were grown to confluence in 10 cm culture dishes. The 2×10⁵ GFP+ control or JAG1 OE cells were seeded onto the plate in osteoblast media. Cell sorting was performed to purify the GFP-negative MC3T3-E1 osteoblasts 5 days after initial coculture. RNA from FACS-separated MC3T3-E1 cells was collected in RLT lysis buffer, extracted with RNeasy Mini Kit (QIAGEN), and subjected to quantitative RT-PCR.

For microarray analysis the quality of the FACS-separated MC3T3-E1 RNA samples was monitored using the 2100 bioanalyzer (Agilent) before gene expression profiling with the Agilent mouse 4344k microarrays. To find genes regulated by JAGGED 1 and MRK-003 in osteoblasts, expression data of MC3T3-E1 under the indicated coculture and treatment conditions were generated and normalized by the array median, and probes were filtered by the expression levels. Probes with >2-fold changes in MC3T3-E1 cells cocultured with JAG1 OE tumor cells relative to vector-control tumor cells were identified as the regulated genes.

Osteoclastogenesis Coculture Assay

After seeding 5×10⁴ control or JAG1 OE tumor cells/well into 12-well plates, murine pre-osteoclast Raw 264.7 (2×10⁵ cells/well) or MOCP5 (5×10⁵ cells/well) cells in media containing 30 ng/ml RANKL and DMSO or 1 μM MRK-003 were added the next day. Media were changed every 2 days. TRAP staining was performed on day 6 using a leukocyte acid phosphatase kit (Sigma). TRAP⁺-multinucleated cells were scored as mature osteoclasts. The number of nuclei per osteoclast was quantified using TRAP-stained images. Mouse specific qRT-PCR primers were used to selectively quantify Raw264.7 osteoclast gene expression levels after 6 days of coculture (see Table 1 below).

For primary osteoclast coculture assays, bone marrow cells were flushed out from femora and tibiae of 4- to 6-week-old wild-type FVB mice and plated in basal culture medium overnight. The next day, nonadherent cells were added at 1×10⁶/well to 12-well plates that were previously seeded with either control or JAG1 OE tumor cells supplemented with 50 ng/ml RANKL and 50 ng/ml M-CSF. Medium was changed every 3 days. TRAP staining and scoring were performed on days 10-12.

Statistical Analysis

Results are presented as average±standard deviation (SD) or as average±standard error of the mean (SEM), as indicated in figure legends. Comparisons between Kaplan-Meier curves were performed using the log rank test. BLI signals were analyzed by unpaired, two-sided, independent Student's t test without equal variance assumption, nonparametric Mann-Whitney test, or ANOVA. All other comparisons were analyzed by unpaired, two-sided, independent Student's t test without equal variance assumption.

Generation of Knockdown and Overexpression Cells

Stable shRNA-mediated knockdown was achieved with the pSuper-Retro system (OligoEngine) targeting the sequence 5′-CGTACAAGTAGTTCTGTAT-3′ for JAG1 [SEQ ID NO: 1]. shRNA retroviral vectors were transfected into the packaging cell line H29. After 48 hours viruses were collected, filtered and used to infect target cells in the presence of 5 μg/ml polybrene. The infected cells were selected with 1 μg/ml puromycin. For stable overexpression in the SCP28 human breast cancer cell line, human JAGGED1 cDNA was PCR amplified using the primer pair: 5′-ATCCTCGAGAGCACCAGCGCGAACAGCAG-3′ (Sense) [SEQ ID NO: 2] and 5′-ATCGAATTCCCCGCGGTCTGCTATACGAT-3′ (Antisense) [SEQ ID NO: 3]and cloned into the retroviral expression vector pMSCVpuro using XhoI and EcoRI restriction sites (underlined=restriction sites, bold=human JAG1-specific sequences). To overexpress human JAGGED1 in the previously reported SMAD4 KD cells (Kang et al., 2005, which is incorporated herein by reference as if fully set forth), the coding sequence was subcloned from pMSCVpuro-JAGGED1 into the retroviral expression vector pMSCVhygro to allow for double antibiotic selection. For stable overexpression in the TM40D-MB murine breast cancer cell line, mouse Jagged 1 cDNA was PCR amplified using the primer pair: 5′-ATCCTCGAGGTCCGGAGTGCCCGT-3′ (Sense) [SEQ ID NO: 4] and 5′-ATCGAATTCGCAGCCCACTGTCTGCTATAC-3′ (Antisense) [SEQ ID NO: 5] and cloned into the retroviral expression vector pMSCVpuro using XhoI and EcoRI restriction sites (underlined=restriction sites, bold=mouse Jag1-specific sequences). Viruses were generated and used to infect target cells as above and then subsequently selected with 1 μg/ml puromycin or 500 μg/ml hygromycin. Control cell lines were derived from parental vectors alone. In order to avoid clonal variations, a pooled population of at least 500 independent clones of each transfection/transduction was used to generate each stable cell line. The generation of the SMAD4-inducible SCP28-SMAD4Tet cell line is previously described (Korpal et al., 2009, which is incorporated herein by reference as if fully set forth).

Cell Culture

SCP2, SCP28, and 1833 sublines were derived from the parental cell line MDA-MB-231 (American Type Culture Collection, ATCC) (Kang et al., 2003, which is incorporated herein by reference as if fully set forth). These sublines and their genetically modified variants were maintained in Dulbecco's modified Eagle's medium (DMEM, Invitrogen) with 10% fetal bovine serium (FBS), penicillin/streptomycin (GIBCO), fungizone and appropriate selection drugs for transfected plasmids. 67NR, 168FARN, 4T07, 66cl4, and 4T1 were maintained in DMEM with 10% FBS and antibiotics. TM40D-MB murine breast cancer cell line was maintained in DMEM/F12 with 2% FBS, epidermal growth factor, insulin, and antibiotics. H29 cells, a packaging cell line for retrovirus production, were maintained in DMEM supplemented with 10% FBS, 2 mM L-glutamine, and antibiotics. The murine osteoblast cell line MC3T3-E1 subclone 4 (ATCC) and the murine pre-osteoclast cell line MOCP5 was maintained in growth medium αMEM supplemented with 10% FBS and antibiotics. The murine pre-osteoclast cell line Raw 264.7 was maintained in DMEM with 10% FBS and antibiotics for regular culture and supplemented with 30 ng/ml RANKL for osteoclastogenesis assays. The WI38 and BJ human fibroblast cell lines were maintained in Eagle's MEM with 10% FBS, 2 mM L-glutamine, NEAA, and antibiotics. Primary bone marrow cells were flushed from tibias of 4-6 week old wild-type FVB mice, filtered through a 70 μM cell-strainer, and maintained in growth medium αMEM supplemented with 10% FBS and antibiotics. For osteoblast coculture assays, the primary bone marrow cells were maintained in growth medium supplemented with L-ascorbic acid to promote differentiation. For osteoclast coculture assays, primary bone marrow cells were plated for 24 h, after which the non-adherent cells were collected and cultured in M-CSF (50 ng/mL) for 2 days and then RANKL (50 ng/mL) for an additional 3-4 days.

X-Ray Analysis and Quantification

Osteolysis was assessed by X-ray radiography. Anesthetized mice were placed on single wrapped films (X-OMAT AR, Eastman Kodak) and exposed to X-ray radiography at 35 kV for 15 s using a MX-20 Faxitron instrument. Films were developed using a Konica SRX-101A processor. Osteolytic lesions were identified on radiographs as demarcated radiolucent lesions in the bone and quantified using the ImageJ software (National Institutes of Health).

Histomorphometric Analysis and Immunohistochemical Staining

Hindlimb bones were excised from mice at the end point of each experiment, immediately after the last BLI time point. Following this, the tumor-bearing hind limb bones were fixed in 10% neutral-buffered formalin, decalcified in 10% EDTA for 2 weeks, and embedded in paraffin for hematoxylin and eosin (H&E), tartrate-resistant acid phosphatase (TRAP) (Kos et al., 2003, which is incorporated herein by reference as if fully set forth), or immunohistochemical staining. Histomorphometric analysis was performed on H&E stained bone metastasis samples using the Zeiss Axiovert 200 microscope and the AxioVision software version 4.6.3 SP1. For quantitative analysis of lesion area, a 5× objective was used to focus on the tumor region of interest and images were acquired using the AxioCamICc3 camera set to an exposure of 100 ms. Lesions that were larger than the field of view were quantified by acquiring multiple images to encompass the entire lesion. The “spline” function of the AxioVision software was used to outline the region of interest and subsequently quantify the lesion area. Osteoclast number was assessed as multinucleated TRAP+ cells along the tumor-bone interface and reported as number/mm of interface (Yin et al., 2003, which is incorporated herein by reference as if fully set forth). Immunohistochemical analysis was performed with heat-induced antigen retrieval. Primary antibodies used were anti-JAGGED1 (Santa Cruz, sc-6011) and anti-Ki67 (Dako, Denmark). Biotinylated secondary antibody was used with Vectastain ABC Kit (Vector Laboratories) and DAB detection kit (Zymed) to reveal the positively stained cells with nuclei counterstained with hematoxylin.

Notch Reporter and siRNA Transfection Assays

For transfection experiments, MC3T3-E1 osteoblasts were seeded at 2×10⁵ cells/well in 12-well plates and grown to 95% confluence. For reporter assays, the firefly luciferase Notchreporter (Zeng et al., 2005, which is incorporated herein by reference as if fully set forth) and cytomegalovirus (CMV)-Renilla luciferase control (Promega) plasmids were transfected using Lipofectamine 2000 at concentrations designated by the manufacturer's instructions. After 4 hours, the transfection media was changed to regular media containing 1×10⁵ vector control or JAG1 OE tumor cells per well and plated in triplicate in the presence of DMSO or MRK-003. Following 2 days, the coculture was lysed and subjected to a luciferase assay in which firefly counts (Notch reporter activity) was divided by renilla counts to normalize for transfection efficiency. For siRNA transfection experiments, a scrambled control siRNA or two distinct targeting siRNAs against Rbpj #1—GCACAGAAGUCUUACGGAAAUGAAA [SEQ ID NO: 6] and #2—CCAUUACGGGCAGACUGUCAAGCUU [SEQ ID NO: 7] or Hey 1 #1—GCAGCAAACCUUGGCAAGCCCUAUA [SEQ ID NO: 8] and #2—UCACCCAGACUACAGCUCCUCAGAU [SEQ ID NO: 9] (Invitrogen Stealth RNAi) were transfected into MC3T3-E1 osteoblasts using Lipofectamine 2000 at concentrations designated by the manufacturers instructions. After 4 hours, the transfection media was changed to regular media containing 1×10⁴ vector control or JAG1 OE tumor cells per well and plated in triplicate. Following 6 days, the coculture was lysed and subjected to a luciferase assay to selectively quantify the number of tumor cells. For gene expression analysis, RNA from cocultures was collected in RLT lysis buffer, extracted with RNeasy mini kit (Qiagen), and subjected to quantitative RT-PCR.

Transwell Invasion Assays.

Control or JAG1 OE tumor cells were resuspended at 1×10⁵ cells in serum-free media and placed in inserts (Costar) containing 8-μm pores with matrigel (1 mg/ml). These inserts were placed in wells that contained media with serum. 12 h post-seeding, serum-containing media was aspirated, and 500 μl of trypsin was placed into the wells to trypsinize the cells that had passed through the pores. Trypsin was neutralized with serum-containing media and centrifuged for 2 min at 1000 rpm. 900 μl of media was aspirated and the cell pellet was resuspended in the remaining 100 μl. 10 μl of this mixture was used to count the number of cells that had migrated using a hemacytometer.

Western Blot Analyses

SDS lysis buffer (0.05 mM Tris-HCl, 50 mM BME, 2% SDS, 0.1% Bromophenol blue, 10% glycerol) was used to collect protein from cultured cells. Heat denatured protein was then equally loaded, separated on an SDS-page gel, transferred onto a pure nitrocellulose membrane (BioRad), and blocked with either 5% milk or 5% BSA. Primary antibodies for immunoblotting included: goat anti-JAGGED1 (1:1000 dilution, sc-6011, Santa Cruz), rabbit anti-phosho-SMAD2 (1:1000 dilution, Ser465/467, Cell Signaling), and mouse anti-β-actin (1:4000 dilution, Abcam) for loading control. Membranes were incubated with horseradish peroxidase (HRP)-conjugated anti-mouse secondary antibody (1:2000 dilution, GE Healthcare) or anti-rabbit secondary antibody (1:2000 dilution, GE Healthcare) for 1 h and chemiluminescence signals were detected by ECL substrate (GE Healthcare).

Gene Set Enrichment Analysis

GSEA v2.0 (Subramanian et al., 2005, which is incorporated herein by reference as if fully set forth, was used). Normalized microarray expression data (Kang et al., 2003, which is incorporated herein by reference as if fully set forth) of weakly and strongly bone metastatic lines were rank-ordered by expression using the provided signal-to-noise metric. Multiple probe matches for the same gene were collapsed into one value, with the highest probe reading being used in each case. TGFβ response gene sets were generated by taking the top 100 genes from the previously published TGFβ response signature of MDA-MB-231 (Padua et al., 2008, which is incorporated herein by reference as if fully set forth). Gene sets were tested for enrichment in rank ordered lists via GSEA using a weighted statistic and compared to enrichment results from 1000 random permutations of the gene set to obtain p-values.

Pharmacological Inhibitor MRK-003

MRK-003 is a potent and specific gamma-secretase inhibitor whose biochemical, cellular and pharmacological properties have been extensively studied and reported. MRK-003 is a cyclic sulfamide with sub-nanomolar potency inhibiting gamma-secretase-mediated cleavage of Notch to its active form (NICD) (Lewis et al., 2007, which is incorporated herein by reference as if fully set forth). Cell-based studies of the mechanism of action and exposure/efficacy experiments revealed that continuous exposure to MRK-003 is not required for maximal activity, since 48 hours of target engagement is sufficient to induce potent Notch inhibition (Tammam et al., 2009, which is incorporated herein by reference as if fully set forth). Further, pharmacokinetic and pharmacodynamic studies in mice indicate that intermittent exposure is also sufficient to produce robust efficacy (Tammam et al., 2009, which is incorporated herein by reference as if fully set forth). Importantly, the dosing “holiday” also allows for recovery from transient intestinal metaplasia (goblet cell induction) that results from Notch inhibition. These preclinical findings have translated into the clinic, as once-weekly dosing (intermittent exposure) was well-tolerated and produced strong clinical responses (LoRusso et al, AACR 2009, which is incorporated herein by reference as if fully set forth). For xenograft experiments, mice were administered the vehicle (0.5% methylcellulose) or MRK-003 by oral gavage twice a week at a 100 mg/kg dosage. The dosing schedule was 2-days on, 5-days off. MRK-003 was dissolved in DMSO for in vitro studies.

Pharmacological Inhibitors, Neutralizing Antibodies, and Recombinant Proteins

For in vitro experiments, GSI-IX (Calbiochem) and TGFβ Receptor 1 (EMD Biosciences 616451) were dissolved in DMSO. Mammalian cancer cells were seeded on a 12-well plate and treated with either DMSO or EMD616451 at time 0. Cells are then treated with recombinant TGFβ1 (R&D Systems) for the indicated duration of time. RNA and protein were collected and analyzed for JAGGED1 expression as described above. For in vivo experiments, TGFβ Receptor 1 kinase inhibitor (LY2109761, Eli Lilly) was dissolved in NaCMC 1% w/w/SLS 0.5%/Antifoam 0.05% at a concentration of 15 g/L (Korpal et al., 2009, which is incorporated herein by reference as if fully set forth). Bone metastasis samples were collected from mice inoculated with SCP28 breast cancer cells and treated with either the solvent control or LY2109761 TGF-βR1 kinase inhibitor as previously reported in (Korpal et al., 2009, which is incorporated herein by reference as if fully set forth). RNA analysis of the in vivo samples was performed as described above. Anti-murine IL-6 antibody (MBL) was administered at a concentration of 0.5 and 1.0 μg/ml. Recombinant rat JAGGED1/Fc chimera (R&D systems) was dissolved in PBS and plated at a concentration of 0.5 μg/ml in 12-well plates that had been pre-coated with anti-Fc antibody for 1 hour and blocked with DMEM containing 10% FBS for 2 hours. Recombinant human IL-6 (R&D systems) was dissolved in PBS containing 0.1% FBS and administered at a concentration of 10 and 100 ng/ml. Recombinant human TGFβ1 (R&D systems) was dissolved in PBS and administered at a concentration of 100 pM.

Murine IL-6 ELISA

Quantitative levels of murine IL-6 in the conditioned medium of cultured and cocultured cells were determined in triplicate by ELISA according to the manufacturer's protocol (Quantikine immunoassay kit, R&D systems).

Quantitative RT-PCR

RNA from in vitro cultured cells or flow cytometry-separated cells was collected in RLT lysis buffer and extracted with RNeasy mini kit (Qiagen). RNA extraction from in vivo tissue samples was performed using Trizol (Invitrogen) according to the manufacturer's protocol. cDNA synthesis of RNA was performed using Superscript III First-Strand (Invitrogen). Quantitative RT-PCR was performed using Power Syber Green PCR Master Mix (Applied Biosystems) with the ABI Prism 7900HT thermocycler (Applied Biosystems) according to the manufacturer's protocol. A standard curve for each gene was generated by serial dilutions of a standard. Values were then normalized by the amount of GAPDH or @-actin in each sample. For in vivo samples, species-specific primers were employed for gene expression analysis in the tumor compartment (human) versus stroma compartment (mouse). Primer sequences are reported listed in the following table.

TABLE 1

| Gene | Species | Forward | Reverse |
|---|---|---|---|
| JAG1 | Human | GAGCTATTTGCCGACAAGGC [SEQ ID NO: 10] | GGAGTTTGCAAGACCCATGC [SEQ ID NO: 11] |
| GAPDH | Human | GGAGTCAACGGATTTGGTCGTA [SEQ ID NO: 12] | GGCAACAATATCCACTTTACCAGAGT [SEQ ID NO: 13] |
| Jag 1 | Mouse | ACACAGGGATTGCCCACTTC [SEQ ID NO: 14 | AGCCAAAGCCATAGTAGTGGTCAT [SEQ ID NO: 15] |
| Dll1 | Mouse | GCGAGCTGCACGGACCTTGA [SEQ ID NO: 16] | GCCCAAGGGGCAATGGCAGG [SEQ ID NO: 17] |
| Notch1 | Mouse | CCAGCAGATGATCTTCCCGTAC [SEQ ID NO: 18] | TAGACAATGGAGCCACGGATGT [SEQ ID NO: 19] |
| Notch2 | Mouse | TCTATCCCCCGTCGATTCG [SEQ ID NO: 20] | GATGTGATCATGGGAGAGGATGT [SEQ ID NO: 21] |
| Notch3 | Mouse | CCAGGGAATTTCAGGTGCAT [SEQ ID NO: 22] | GCCGTCGAGGCAAGAACA [SEQ ID NO: 23] |
| Notch4 | Mouse | GAGGACCTGGTTGAAGAATTGATC [SEQ ID NO: 24] | TGCAGTTTTTCCCCTTTTATCC [SEQ ID NO: 25] |
| Hes1 | Mouse | CCCCAGCCAGTGTCAACAC [SEQ ID NO: 26] | TGTGCTCAGAGGCCGTCTT [SEQ ID NO: 27] |
| Hes2 | Mouse | GCTACCGGACCAAGGAAGTTC [SEQ ID NO: 28] | GAGCTAGACTGTTCTCAAAGTGAGTGA [SEQ ID NO: 29] |
| Hes3 | Mouse | AAGGGAGCAGAAAAGCATCA [SEQ ID NO: 30] | CTATGGCAGGGAGCTTTGAG [SEQ ID NO: 31] |
| Hes5 | Mouse | TGGGCACATTTGCCTTTTGT [SEQ ID NO: 32] | CAGGCTGAGTGCTTTCCTATGA [SEQ ID NO: 33] |
| Hey1 | Mouse | GGGAGGGTCAGCAAAGCA [SEQ ID NO: 34] | GCTGCGCATCTGATTTGTCA [SEQ ID NO: 35] |
| Hey2 | Mouse | CACATCAGAGTCAACCCCATGT [SEQ ID NO: 36] | GTGAGGAGAGCAGAGCCATGA [SEQ ID NO: 37] |
| HeyL | Mouse | AGATGCAAGCCCGGAAGAA [SEQ ID NO: 38] | CGCAATTCAGAAAGGCTACTGTT [SEQ ID NO: 39] |
| TGFβ1 | Mouse | TGGAGCCTGGACACACAGTA [SEQ ID NO: 40] | TGTGTTGGTTGTAGAGGGCA [SEQ ID NO: 41] |
| Runx2 | Mouse | AAATGCCTCCGCTGTTATGAA [SEQ ID NO: 42] | GCTCCGGCCCACAAATCT [SEQ ID NO: 43] |
| Osx | Mouse | CCCTTCTCAAGCACCAATGG [SEQ ID NO: 44] | AGGGTGGGTAGTCATTTGCATAG [SEQ ID NO: 45] |
| AcpS | Mouse | CACTCCCACCCTGAGATTTGTG [SEQ ID NO: 46] | ACGGTTCTGGCGATCTCTTTG [SEQ ID NO: 47] |
| Rankl | Mouse | CAGGTTTGCAGGACTCGAC [SEQ ID NO: 48] | AGCAGGGAAGGGTTGGACA [SEQ ID NO: 49] |
| Nfatc | Mouse | AAGTCTCACCACAGGGCTCACT [SEQ ID NO: 50] | CAAGTAACCGTGTAGCTGCACAAT [SEQ ID NO: 51] |
| c-Myc | Mouse | TGAGCCCCTAGTGCTGCAT [SEQ ID NO: 52] | TCCACAGACACCACATCAATTTC [SEQ ID NO: 53] |
| c-Src | Mouse | CTCCCGCACCCAGTTCAA [SEQ ID NO: 54] | GCCATCAGCATGTTTGGAGTAGT [SEQ ID NO: 55] |
| Mmp9 | Mouse | GTTTTTGATGCTATTGCTGAGATCCA [SEQ ID NO: 56] | CCCACATTTGACGTCCAGAGAAGAA [SEQ ID NO: 57] |
| Car2 | Mouse | CGTCCAAGAGCATTGTCAACA [SEQ ID NO: 58] | CCTCCTTTCAGCACTGCATTG [SEQ ID NO: 59] |
| Itgb3 | Mouse | CCTTTGCCCAGCCTTCCA [SEQ ID NO: 60] | GTCCCCACAGTTACATTG [SEQ ID NO: 61] |
| Ctsk | Mouse | AGAGAGCAGTGGCGCGGGTA [SEQ ID NO: 62] | CCAGCTCTCTCCCCAGCTGTT [SEQ ID NO: 63] |
| Tm7sf4 | Mouse | TGGGTGCTGTTTGCCGCTGT [SEQ ID NO: 64] | TGGGTTCCTTGCTTCTCTCCACG [SEQ ID NO: 65] |
| Jundam2 | Mouse | TGCGCCCTTGCACTTCCTGG [SEQ ID NO: 66] | GCCGCTCTGACTCCCTCTGC [SEQ ID NO: 67] |
| Gapdh | Mouse | TCCCACTCTTCCACCTTCGATGC [SEQ ID NO: 68] | GGGTCTGGGATGGAAATTGTGAGG [SEQ ID NO: 69] |
| β-actin | Mouse | TCCTCCTGAGCGCAAGTACTCT [SEQ ID NO: 70] | CGGACTCATCGTACTCCTGCTT [SEQ ID NO: 71] |

The results contained herein were later reported in Sethi, N., Dai, X., Winter, C. and Kang, Y. (2011). Tumor-Derived Jagged1 Promotes Osteolytic Bone Metastasis of Breast Cancer by Engaging Notch Signalling in Bone Cells. Cancer Cell 2, 192-205, which is incorporated herein by reference as if fully set forth.

### REFERENCES

[1] Ara, T., Song, L., Shimada, H., Keshelava, N., Russell, H. V., Metelitsa, L. S., Groshen, S. G., Seeger, R. C., and DeClerck, Y. A. (2009). Interleukin-6 in the bone marrow microenvironment promotes the growth and survival of neuroblastoma cells. Cancer Res. 69, 329-337.
[2] Artavanis-Tsakonas, S., Rand, M. D., and Lake, R. J. (1999). Notch signaling: cell fate control and signal integration in development. Science 284, 770-776.
[3] Aslakson, C. J., and Miller, F. R. (1992). Selective events in the metastatic process defined by analysis of the sequential dissemination of subpopulations of a mouse mammary tumor. Cancer Res. 52, 1399-1405.
[4] Bai, S., Kopan, R., Zou, W., Hilton, M. J., Ong, C. T., Long, F., Ross, F. P., and Teitelbaum, S. L. (2008). NOTCH1 regulates osteoclastogenesis directly in osteoclast precursors and indirectly via osteoblast lineage cells. J. Biol. Chem. 283, 6509-6518.
[5] Calvi, L. M., Adams, G. B., Weibrecht, K. W., Weber, J. M., Olson, D. P., Knight, M. C., Martin, R. P., Schipani, E., Divieti, P., Bringhurst, F. R., et al. (2003). Osteoblastic cells regulate the haematopoietic stem cell niche. Nature 425, 841-846.
[6] de la Mata, J., Uy, H. L., Guise, T. A., Story, B., Boyce, B. F., Mundy, G. R., and Roodman, G. D. (1995). Interleukin-6 enhances hypercalcemia and bone resorption mediated by parathyroid hormone-related protein in vivo. J. Clin. Invest. 95, 2846-2852.
[7] Dickson, B. C., Mulligan, A. M., Zhang, H., Lockwood, G., O'Malley, F. P., Egan, S. E., and Reedijk, M. (2007). High-level JAG1 mRNA and protein predict poor outcome in breast cancer. Mod. Pathol. 20, 685-693.
[8] Engin, F., Yao, Z., Yang, T., Zhou, G., Bertin, T., Jiang, M. M., Chen, Y., Wang, L., Zheng, H., Sutton, R. E., et al. (2008). Dimorphic effects of Notch signaling in bone homeostasis. Nat. Med. 14, 299-305.
[9] Fukushima, H., Nakao, A., Okamoto, F., Shin, M., Kajiya, H., Sakano, S., Bigas, A., Jimi, E., and Okabe, K. (2008). The association of Notch2 and NF-kappaB accelerates RANKL-induced osteoclastogenesis. Mol. Cell. Biol. 28, 6402-6412.
[10] Guise, T. A., Yin, J. J., Taylor, S. D., Kumagai, Y., Dallas, M., Boyce, B. F., Yoneda, T., and Mundy, G. R. (1996). Evidence for a causal role of parathyroid hormone-related protein in the pathogenesis of human breast cancer-mediated osteolysis. J. Clin. Invest. 98, 1544-1549.
[11] Han, J., Ma, I., Hendzel, M. J., and Allalunis-Turner, J. (2009). The cytotoxicity of gamma-secretase inhibitor I to breast cancer cells is mediated by proteasome inhibition, not by gamma-secretase inhibition. Breast Cancer Res. 11, R57.
[12] Hilton, M. J., Tu, X., Wu, X., Bai, S., Zhao, H., Kobayashi, T., Kronenberg, H. M., Teitelbaum, S. L., Ross, F. P., Kopan, R., and Long, F. (2008). Notch signaling maintains bone marrow mesenchymal progenitors by suppressing osteoblast differentiation. Nat. Med. 14, 306-314.
[13] Jhappan, C., Gallahan, D., Stahle, C., Chu, E., Smith, G. H., Merlino, G., and Callahan, R. (1992). Expression of an activated Notch-related int-3 transgene interferes with cell differentiation and induces neoplastic transformation in mammary and salivary glands. Genes Dev. 6, 345-355.
[14] Kang, Y., Siegel, P. M., Shu, W., Drobnjak, M., Kakonen, S. M., Cordon-Cardo, C., Guise, T. A., and Massague, J. (2003). A multigenic program mediating breast cancer metastasis to bone. Cancer Cell 3, 537-549.
[15] Kang, Y., He, W., Tulley, S., Gupta, G. P., Serganova, I., Chen, C. R., Manova-Todorova, K., Blasberg, R., Gerald, W. L., and Massague, J. (2005). Breast cancer bone metastasis mediated by the Smad tumor suppressor pathway. Proc. Natl. Acad. Sci. USA 102, 13909-13914.
[16] Kopan, R., and Ilagan, M. X. (2009). The canonical Notch signaling pathway: unfolding the activation mechanism. Cell 137, 216-233.
[17] Korpal, M., Yan, J., Lu, X., Xu, S., Lerit, D. A., and Kang, Y. (2009). Imaging transforming growth factor-beta signaling dynamics and therapeutic response in breast cancer bone metastasis. Nat. Med. 15, 960-966.
[18] Lelekakis, M., Moseley, J. M., Martin, T. J., Hards, D., Williams, E., Ho, P., Lowen, D., Javni, J., Miller, F. R., Slavin, J., and Anderson, R. L. (1999). A novel orthotopic model of breast cancer metastasis to bone. Clin. Exp. Metastasis 17, 163-170.
[19] Leong, K. G., Niessen, K., Kulic, I., Raouf, A., Eaves, C., Pollet, I., and Karsan, A. (2007). Jagged1-mediated Notch activation induces epithelial-to-mesenchymal transition through Slug-induced repression of E-cadherin. J. Exp. Med. 204, 2935-2948.
[20] Li, Z., Schem, C., Shi, Y. H., Medina, D., and Zhang, M. (2008). Increased COX2 expression enhances tumor-induced osteoclastic lesions in breast cancer bone metastasis. Clin. Exp. Metastasis 25, 389-400.
[21] Minn, A. J., Gupta, G. P., Siegel, P. M., Bos, P. D., Shu, W., Giri, D. D., Viale, A., Olshen, A. B., Gerald, W. L., and Massague, J. (2005). Genes that mediate breast cancer metastasis to lung. Nature 436, 518-524.
[22] Mitsiades, C. S., Mitsiades, N. S., Munshi, N. C., Richardson, P. G., and Anderson, K. C. (2006). The role of the bone microenvironment in the pathophysiology and therapeutic management of multiple myeloma: interplay of growth factors, their receptors and stromal interactions. Eur. J. Cancer 42, 1564-1573.
[23] Mundy, G. R. (2002). Metastasis to bone: causes, consequences and therapeutic opportunities. Nat. Rev. Cancer 2, 584-593.
[24] Niranjan, T., Bielesz, B., Gruenwald, A., Ponda, M. P., Kopp, J. B., Thomas, D. B., and Susztak, K. (2008). The Notch pathway in podocytes plays a role in the development of glomerular disease. Nat. Med. 14, 290-298.
[25] Nobta, M., Tsukazaki, T., Shibata, Y., Xin, C., Moriishi, T., Sakano, S., Shindo, H., and Yamaguchi, A. (2005). Critical regulation of bone morphogenetic protein-induced osteoblastic differentiation by Delta 1/Jagged1-activated Notch1 signaling. J. Biol. Chem. 280, 15842-15848.
[26] Pacifici, R. (2010). The immune system and bone. Arch. Biochem. Biophys. 503, 41-53.
[27] Reedijk, M., Odorcic, S., Chang, L., Zhang, H., Miller, N., McCready, D. R., Lockwood, G., and Egan, S. E. (2005). High-level coexpression of JAG1 and NOTCH1 is observed in human breast cancer and is associated with poor overall survival. Cancer Res. 65, 8530-8537.
[28] Rizzo, P., Osipo, C., Foreman, K., Golde, T., Osborne, B., and Miele, L. (2008). Rational targeting of Notch signaling in cancer. Oncogene 27, 5124-5131.
[29] Salgado, R., Junius, S., Benoy, I., Van Dam, P., Vermeulen, P., Van Marck, E., Huget, P., and Dirix, L. Y. (2003). Circulating interleukin-6 predicts survival in patients with metastatic breast cancer. Int. J. Cancer 103, 642-646.
[30] Santagata, S., Demichelis, F., Riva, A., Varambally, S., Hofer, M. D., Kutok, J. L., Kim, R., Tang, J., Montie, J. E., Chinnaiyan, A. M., et al. (2004). JAGGED1 expression is associated with prostate cancer metastasis and recurrence. Cancer Res. 64, 6854-6857.
[31] Sasser, A. K., Sullivan, N. J., Studebaker, A. W., Hendey, L. F., Axel, A. E., and Hall, B. M. (2007). Interleukin-6 is a potent growth factor for ER-alpha-positive human breast cancer. FASEB J. 21, 3763-3770.
[32] Sethi, N., and Kang, Y. (2011). Dysregulation of developmental pathways in bone metastasis. Bone 48, 16-22.
[33] Tezuka, K., Yasuda, M., Watanabe, N., Morimura, N., Kuroda, K., Miyatani, S., and Hozumi, N. (2002). Stimulation of osteoblastic cell differentiation by Notch. J. Bone Miner. Res. 17, 231-239.
[34] Wang, Y., Klijn, J. G., Zhang, Y., Sieuwerts, A. M., Look, M. P., Yang, F., Talantov, D., Timmermans, M., Meijer-van Gelder, M. E., Yu, J., et al. (2005). Gene-expression profiles to predict distant metastasis of lymph-node-negative primary breast cancer. Lancet 365, 671-679.
[35] Wang, Z., Banerjee, S., Li, Y., Rahman, K. M., Zhang, Y., and Sarkar, F. H. (2006). Down-regulation of notch-1 inhibits invasion by inactivation of nuclear factor-kappaB, vascular endothelial growth factor, and matrix metalloproteinase-9 in pancreatic cancer cells. Cancer Res. 66, 2778-2784.
[36] Weber, J. M., and Calvi, L. M. (2010). Notch signaling and the bone marrow hematopoietic stem cell niche. Bone 46, 281-285.
[37] Weber, J. M., Forsythe, S. R., Christianson, C. A., Frisch, B. J., Gigliotti, B. J., Jordan, C. T., Milner, L. A., Guzman, M. L., and Calvi, L. M. (2006). Parathyroid hormone stimulates expression of the Notch ligand Jagged1 in osteoblastic cells. Bone 39, 485-493.
[38] Xu, Z., Hurchla, M. A., Deng, H., Uluckan, O., Bu, F., Berdy, A., Eagleton, M. C., Heller, E. A., Floyd, D. H., Dirksen, W. P., et al. (2009). Interferon-gamma targets cancer cells and osteoclasts to prevent tumor-associated bone loss and bone metastases. J. Biol. Chem. 284, 4658-4666.
[39] Yamada, T., Yamazaki, H., Yamane, T., Yoshino, M., Okuyama, H., Tsuneto, M., Kurino, T., Hayashi, S., and Sakano, S. (2003). Regulation of osteoclast development by Notch signaling directed to osteoclast precursors and through stromal cells. Blood 101, 2227-2234.
[40] Yauch, R. L., Gould, S. E., Scales, S. J., Tang, T., Tian, H., Ahn, C. P., Marshall, D., Fu, L., Januario, T., Kallop, D., et al. (2008). A paracrine requirement for hedgehog signalling in cancer. Nature 455, 406-410.
[41] Yin, J. J., Selander, K., Chirgwin, J. M., Dallas, M., Grubbs, B. G., Wieser, R., Massague, J., Mundy, G. R., and Guise, T. A. (1999). TGF-beta signaling blockade inhibits PTHrP secretion by breast cancer cells and bone metastases development. J. Clin. Invest. 103, 197-206.
[42] Zanotti, S., and Canalis, E. (2010). Notch and the skeleton. Mol. Cell. Biol. 30, 886-896.
[43] Zavadil, J., Cermak, L., Soto-Nieves, N., and Bottinger, E. P. (2004). Integration of TGF-beta/Smad and Jagged1/Notch signalling in epithelial-to-mesenchymal transition. EMBO J. 23, 1155-1165.
[44] Zeng, Q., Li, S., Chepeha, D. B., Giordano, T. J., Li, J., Zhang, H., Polverini, P. J., Nor, J., Kitajewski, J., and Wang, C. Y. (2005). Crosstalk between tumor and endothelial cells promotes tumor angiogenesis by MAPK activation of Notch signaling. Cancer Cell 8, 13-23.
[45] Zhang, X. H., Wang, Q., Gerald, W., Hudis, C. A., Norton, L., Smid, M., Foekens, J. A., and Massague, J. (2009). Latent bone metastasis in breast cancer tied to Src-dependent survival signals. Cancer Cell 16, 67-78.
[46] Kos, C. H., Karaplis, A. C., Peng, J. B., Hediger, M. A., Goltzman, D., Mohammad, K. S., Guise, T. A., and Pollak, M. R. (2003). The calcium-sensing receptor is required for normal calcium homeostasis independent of parathyroid hormone. The Journal of clinical investigation 111, 1021-1028.
[47] Lewis, H. D., Leveridge, M., Strack, P. R., Haldon, C. D., O'Neil, J., Kim, H., Madin, A., Hannam, J. C., Look, A. T., Kohl, N., et al. (2007). Apoptosis in T cell acute lymphoblastic leukemia cells after cell cycle arrest induced by pharmacological inhibition of notch signaling. Chemistry & biology 14, 209-219.
[48] Padua, D., Zhang, X. H., Wang, Q., Nadal, C., Gerald, W. L., Gomis, R. R., and Massague, J. (2008). TGFbeta primes breast tumors for lung metastasis seeding through angiopoietin-like 4. Cell 133, 66-77.
[49] Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: A knowledge-based approach for interpreting genome-wide expression profiles. Proceedings of the National Academy of Sciences of the United States of America 102, 15545-15550.
[50] Tammam, J., Ware, C., Efferson, C., O'Neil, J., Rao, S., Qu, X., Gorenstein, J., Angagaw, M., Kim, H., Kenific, C., et al. (2009). Down-regulation of the Notch pathway mediated by a gamma-secretase inhibitor induces anti-tumour effects in mouse models of T-cell leukaemia. British journal of pharmacology 158, 1183-1195.
[51] Yin, J. J., Mohammad, K. S., Kakonen, S. M., Harris, S., Wu-Wong, J. R., Wessale, J. L., Padley, R. J., Garrett, I. R., Chirgwin, J. M., and Guise, T. A. (2003). A causal role for endothelin-1 in the pathogenesis of osteoblastic bone metastases. Proceedings of the National Academy of Sciences of the United States of America 100, 10954-10959.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A method for diagnosing an increased risk of breast cancer bone metastasis in a subject having breast cancer comprising:
obtaining a sample from the subject; and
determining whether the sample has a Jagged1 high level expression marker;
wherein presence of the Jagged1 high level expression marker in the sample indicates the increased risk of having breast cancer bone metastasis for the subject.

2. The method of claim 1 further comprising obtaining a control sample, wherein the determining step includes detecting an amount of Jagged1 in the sample, detecting an amount of Jagged1 in the control sample, and comparing the amount of Jagged1 the sample to the amount of Jagged1 in the control sample, and wherein the amount of Jagged1 in the sample being at least 2-fold greater than the amount of Jagged1 in the control sample is the Jagged1 high level expression marker.

3. The method of claim 2, wherein the detecting includes analysis of the sample and the control sample with a composition including an anti-Jagged1 antibody.

4. The method of claim 3, wherein the sample is a breast tumor sample and the control sample is a non-tumor breast tissue sample.

5. The method of claim 3, wherein the sample is a serum sample from the subject and the control sample is a serum sample from an individual lacking breast cancer bone metastasis.

6. The method of claim 1 further comprising obtaining a control sample, wherein the determining step includes detecting an amount of Jagged1 mRNA in the sample and an amount of Jagged1 mRNA in the control sample, and wherein the amount of Jagged1 mRNA in the sample that is at least 2-fold greater than the amount of Jagged1 mRNA in the control sample is the Jagged1 high level expression marker.

7. The method of claim 6, wherein the sample is a breast tumor sample from the subject and the control sample is a non-tumor breast tissue sample.

8. The method of claim 1 further comprising obtaining a control sample, wherein the determining step includes detecting an amount of Il-6 in the sample, detecting an amount of IL-6 in the control sample, and comparing the amount of IL-6 in the sample to the amount of IL-6 in the control sample, and wherein the amount of IL-6 in the sample being at least 2-fold greater than the amount of IL-6 in the control sample is the Jagged1 high level expression marker.

9. The method of claim 8, wherein the detecting includes ELISA with a composition including an anti-IL-6 antibody.

10. The method of claim 9, wherein the sample is at least one of a serum sample or a bone aspirate from the subject, and the control sample is a serum control sample from an individual lacking breast cancer bone metastasis or a bone aspirate from an individual lacking breast cancer bone metastasis.

11. The method of claim 8, wherein the detecting includes contacting anti-IL-6 antibody to bone aspirates, IL-6 staining of bone marrow, or staining of IL-6 downstream pathway in metastatic tumors; the respective samples are bone aspirates from the subject having breast cancer, bone marrow from the subject having breast cancer, metastatic tumors from the subject having breast cancer; and the respective control samples are bone aspirates from non-metastatic bone, bone marrow from non-metastatic bone, non-tumor breast tissue.

12. The method of claim 1 further comprising diagnosing the subject as having the increased risk of breast cancer bone metastasis upon determining the presence of the Jagged1 high level expression marker in the sample.

13. The method of claim 1 further comprising diagnosing the subject as having decreased sensitivity to RANK or RANKL targeting treatments upon determining the presence of the Jagged1 high level expression marker in the sample.

14. The method of claim 1 further comprising diagnosing the subject as having increased sensitivity to NOTCH targeting treatments upon determining the presence of the Jagged1 high level expression marker in the sample.

15. The method of claim 1 further comprising diagnosing the subject as having increased sensitivity to Jagged1 targeting treatments against breast cancer bone metastasis upon determining the presence of the Jagged1 high level expression marker in the sample.

16. A method of treating a breast cancer patient comprising:
administering to the breast cancer patient at least one therapy selected from the group consisting of Notch targeting treatments and Jagged1 targeting treatments,
wherein the administering occurs after a determination of a presence of a Jagged1 high level expression marker in a sample from the breast cancer patient.

17. The method of claim 16, wherein the determination of the presence of the Jagged1 high level expression marker in the sample from the breast cancer patient is performed by a method comprising:
obtaining the sample from the breast cancer patient; and
determining whether the sample has a Jagged1 high level expression marker;
wherein presence of the Jagged1 high level expression marker in the sample indicates the increased risk of having breast cancer bone metastasis for the breast cancer patient.

18. The method of claim 16, wherein the at least one therapy includes administering at least one agent selected from the group consisting of a Jagged1 activity down regulator, a GSI, a Jagged1 gene expression down regulator, and an RNAi molecule that has a nucleotide sequence complementary to at least a portion of Jagged1 mRNA.

19. The method of claim 16, wherein the at least one therapy includes administering to the breast cancer patient an RNAi molecule having a nucleotide sequence with at least 90% identity to a reference sequence consisting of the RNA sequence corresponding to one of SEQ ID NO: 74, SEQ ID NO: 77, SEQ ID NO: 80, SEQ ID NO: 83, SEQ ID NO: 86, SEQ ID NO: 89., SEQ ID NO: 92, SEQ ID NO: 95, SEQ ID NO: 98, or SEQ ID NO: 101.

20. The method of claim 17, wherein the at least 90% identity is 100% identity.

21. A method of treating a breast cancer patient comprising:
administering to the breast cancer patient at least one therapy selected from the group consisting of RANK targeting treatments and RANKL targeting treatments,
wherein the administering occurs after a determination of an absence of a Jagged1 high level expression marker in a sample from the breast cancer patient.

22. The method of claim 21, wherein the determination of the absence of the Jagged1 high level expression marker in the sample from the breast cancer patient is performed by a method comprising:
obtaining the sample from the breast cancer patient; and
determining whether the sample has a Jagged1 high level expression marker;
wherein presence of the Jagged1 high level expression marker in the sample indicates the increased risk of having breast cancer bone metastasis for the breast cancer patient.

23. The method of claim 21, wherein the at least one therapy includes administering denosumab.

24. A composition comprising at least one agent selected from the group consisting of a Jagged1 activity down regulator, a GSI, a Jagged1 gene expression down regulator, an RNAi molecule that has a nucleotide sequence complementary to at least a portion of Jagged1 mRNA, and a DNA encoding the RNAi molecule that has a nucleotide sequence complementary to at least a portion of Jagged1 mRNA.

25. The composition of claim 24, wherein the at least one agent includes the RNAi molecule or the DNA encoding the RNAi molecule, and the RNAi molecule has a nucleotide sequence having at least 90% identity to a reference sequence consisting of the RNA sequence corresponding to one of SEQ ID NO: 74, SEQ ID NO: 77, SEQ ID NO: 80, SEQ ID NO: 83, SEQ ID NO: 86, SEQ ID NO: 89., SEQ ID NO: 92, SEQ ID NO: 95, SEQ ID NO: 98, or SEQ ID NO: 101.

26. The composition of claim 25, wherein the at least 90% identity is 100% identity.

27. The composition of claim 25 further comprising a pharmaceutically acceptable carrier.

28. The composition of claim 27, wherein the pharmaceutically acceptable carrier includes at least one substance selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.) and phosphate buffered saline (PBS).


SEQUENCE LISTING (107 sequences)

SEQ ID NO: 1 - DNA, 19 bp, Artificial Sequence
  Artificial sequence, JAG1 targeting shRNA
cgtacaagtagttctgtat

SEQ ID NO: 2 - DNA, 29 bp, Artificial Sequence
  Artificial sequence, JAGGED1 primer (sense)
atcctcgagagcaccagcgcgaacagcag

SEQ ID NO: 3 - DNA, 29 bp, Artificial Sequence
  Artificial sequence, JAGGED1 primer (antisense)
atcgaattccccgcggtctgctatacgat

SEQ ID NO: 4 - DNA, 24 bp, Artificial Sequence
  Artificial sequence, Jagged1 primer (sense)
atcctcgaggtccggagtgcccgt

SEQ ID NO: 5 - DNA, 30 bp, Artificial Sequence
  Artificial sequence, Jagged1 primer antisense
atcgaattcgcagcccactgtctgctatac

SEQ ID NO: 6 - RNA, 25 nt, Artificial Sequence
  Artificial sequence, siRNA against Rbpj
gcacagaagucuuacggaaaugaaa

SEQ ID NO: 7 - RNA, 25 nt, Artificial Sequence
  Artificial sequence, siRNA against Rbpj
ccauuacgggcagacugucaagcuu

SEQ ID NO: 8 - RNA, 25 nt, Artificial Sequence
  Artificial sequence, siRNA against Hey1
gcagcaaaccuuggcaagcccuaua

SEQ ID NO: 9 - RNA, 25 nt, Artificial Sequence
  Artificial sequence, siRNA against Hey1
ucacccagacuacagcuccucagau

SEQ ID NO: 10 - DNA, 20 bp, Artificial Sequence
  Artificial sequence, JAG1 human forward primer
gagctatttgccgacaaggc

SEQ ID NO: 11 - DNA, 20 bp, Artificial Sequence
  Artificial sequence, JAG1 human reverse primer
ggagtttgcaagacccatgc

SEQ ID NO: 12 - DNA, 22 bp, Artificial Sequence
  Artificial sequence, GAPDH human forward primer
ggagtcaacggatttggtcgta

SEQ ID NO: 13 - DNA, 26 bp, Artificial Sequence
  Artificial sequence, GAPDH human reverse primer
ggcaacaatatccactttaccagagt

SEQ ID NO: 14 - DNA, 20 bp, Artificial Sequence
  Artificial sequence, Jag1 mouse forward primer
acacagggattgcccacttc

SEQ ID NO: 15 - DNA, 24 bp, Artificial Sequence
  Artificial sequence, Jag1 mouse reverse primer
agccaaagccatagtagtggtcat

SEQ ID NO: 16 - DNA, 20 bp, Artificial Sequence
  Artificial sequence, DII1 mouse forward primer
gcgagctgcacggaccttga

SEQ ID NO: 17 - DNA, 20 bp, Artificial Sequence
  Artificial sequence, DII1 mouse reverse primer
gcccaaggggcaatggcagg

SEQ ID NO: 18 - DNA, 22 bp, Artificial Sequence
  Artificial sequence, Notch1 mouse forward primer
ccagcagatgatcttcccgtac

SEQ ID NO: 19 - DNA, 22 bp, Artificial Sequence
  Artificial sequence, Notch1 mouse reverse primer
tagacaatggagccacggatgt

SEQ ID NO: 20 - DNA, 19 bp, Artificial Sequence
  Artificial sequence, Notch2 mouse forward primer
tctatcccccgtcgattcg

SEQ ID NO: 21 - DNA, 23 bp, Artificial Sequence
  Artificial sequence, Notch2 mouse reverse primer
gatgtgatcatgggagaggatgt

SEQ ID NO: 22 - DNA, 20 bp, Artificial Sequence
  Artificial sequence, Notch3 mouse forward primer
ccagggaatttcaggtgcat

SEQ ID NO: 23 - DNA, 18 bp, Artificial Sequence
  Artificial sequence, Notch3 mouse reverse primer
gccgtcgaggcaagaaca

SEQ ID NO: 24 - DNA, 24 bp, Artificial Sequence
  Artificial sequence, notch4 mouse forward primer
gaggacctggttgaagaattgatc

SEQ ID NO: 25 - DNA, 22 bp, Artificial Sequence
  Artificial sequence, Notch4 mouse reverse primer
tgcagtttttccccttttatcc

SEQ ID NO: 26 - DNA, 19 bp, Artificial Sequence
  Artificial sequence, Hes1 mouse forward primer
ccccagccagtgtcaacac

SEQ ID NO: 27 - DNA, 19 bp, Artificial Sequence
  Artificial sequence, Hes1 mouse reverse primer
tgtgctcagaggccgtctt

SEQ ID NO: 28 - DNA, 21 bp, Artificial Sequence
  Artificial sequence, Hes2 mouse forward primer
gctaccggaccaaggaagttc

SEQ ID NO: 29 - DNA, 27 bp, Artificial Sequence
  Artificial sequence, Hes2 mouse reverse primer
gagctagactgttctcaaagtgagtga

SEQ ID NO: 30 - DNA, 20 bp, Artificial Sequence
  Artificial sequence, Hes3 mouse forward primer
aagggagcagaaaagcatca

SEQ ID NO: 31 - DNA, 20 bp, Artificial Sequence
  Artificial sequence, Hes3 mouse reverse primer
ctatggcagggagctttgag

SEQ ID NO: 32 - DNA, 20 bp, Artificial Sequence
  Artificial sequence, Hes5 mouse forward primer
tgggcacatttgccttttgt

SEQ ID NO: 33 - DNA, 22 bp, Artificial Sequence
  Artificial sequence, Hes5 mouse reverse primer
caggctgagtgctttcctatga

SEQ ID NO: 34 - DNA, 18 bp, Artificial Sequence
  Artificial sequence, Hey1 mouse forward primer
gggagggtcagcaaagca

SEQ ID NO: 35 - DNA, 20 bp, Artificial Sequence
  Artificial sequence, Hey1 mouse reverse primer
gctgcgcatctgatttgtca

SEQ ID NO: 36 - DNA, 22 bp, Artificial Sequence
  Artificial sequence, Hey2 mouse forward primer
cacatcagagtcaaccccatgt

SEQ ID NO: 37 - DNA, 21 bp, Artificial Sequence
  Artificial sequence, Hey2 mouse reverse primer
gtgaggagagcagagccatga

SEQ ID NO: 38 - DNA, 19 bp, Artificial Sequence
  Artificial sequence, HeyL mouse forward primer
agatgcaagcccggaagaa

SEQ ID NO: 39 - DNA, 23 bp, Artificial Sequence
  Artificial sequence, HeyL mouse reverse primer
cgcaattcagaaaggctactgtt

SEQ ID NO: 40 - DNA, 20 bp, Artificial Sequence
  Artificial sequence, TGFB1 mouse forward primer
tggagcctggacacacagta

SEQ ID NO: 41 - DNA, 20 bp, Artificial Sequence
  Artificial sequence, TGFB1 mouse reverse primer
tgtgttggttgtagagggca

SEQ ID NO: 42 - DNA, 21 bp, Artificial Sequence
  Artificial sequence, Runx2 mouse forward primer
aaatgcctccgctgttatgaa

SEQ ID NO: 43 - DNA, 18 bp, Artificial Sequence
  Artificial sequence, Runx2 mouse reverse primer
gctccggcccacaaatct

SEQ ID NO: 44 - DNA, 20 bp, Artificial Sequence
  Artificial sequence, Osx mouse forward primer
cccttctcaagcaccaatgg

SEQ ID NO: 45 - DNA, 23 bp, Artificial Sequence
  Artificial sequence, Osx mouse reverse primer
agggtgggtagtcatttgcatag

SEQ ID NO: 46 - DNA, 22 bp, Artificial Sequence
  Artificial sequence, Acp5 mouse forward primer
cactcccaccctgagatttgtg

SEQ ID NO: 47 - DNA, 21 bp, Artificial Sequence
  Artificial sequence, Acp5 mouse reverse primer
acggttctggcgatctctttg

SEQ ID NO: 48 - DNA, 19 bp, Artificial Sequence
  Artificial sequence, Rankl mouse foward primer
caggtttgcaggactcgac

SEQ ID NO: 49 - DNA, 19 bp, Artificial Sequence
  Artificial sequence, Rankl mouse reverse primer
agcagggaagggttggaca

SEQ ID NO: 50 - DNA, 22 bp, Artificial Sequence
  Artificial sequence - Nfatc mouse forward primer
aagtctcaccacagggctcact

SEQ ID NO: 51 - DNA, 24 bp, Artificial Sequence
  Artificial sequence, Nfatc mouse reverse primer
caagtaaccgtgtagctgcacaat

SEQ ID NO: 52 - DNA, 19 bp, Artificial Sequence
  Artificial sequence, C-myc mouse forward primer
tgagcccctagtgctgcat

SEQ ID NO: 53 - DNA, 23 bp, Artificial Sequence
  Artificial sequence, c-Myc mouse reverse primer
tccacagacaccacatcaatttc

SEQ ID NO: 54 - DNA, 18 bp, Artificial Sequence
  Artificial sequence, c-Src mouse forward primer
ctcccgcacccagttcaa

SEQ ID NO: 55 - DNA, 23 bp, Artificial Sequence
  Artificial sequence, c-Src mouse reverse primer
gccatcagcatgtttggagtagt

SEQ ID NO: 56 - DNA, 26 bp, Artificial Sequence
  Artificial sequence, Mmp9 mouse forward primer
gtttttgatgctattgctgagatcca

SEQ ID NO: 57 - DNA, 25 bp, Artificial Sequence
  Artificial sequence, Mmp9 mouse reverse primer
cccacatttgacgtccagagaagaa

SEQ ID NO: 58 - DNA, 21 bp, Artificial Sequence
  Artificial sequence, Car2 mouse forward primer
cgtccaagagcattgtcaaca

SEQ ID NO: 59 - DNA, 21 bp, Artificial Sequence
  Artificial sequence, Car2 mouse reverse primer
cctcctttcagcactgcattg

SEQ ID NO: 60 - DNA, 18 bp, Artificial Sequence
  Artificial sequence, Itgb3 mouse forward primer
cctttgcccagccttcca

SEQ ID NO: 61 - DNA, 18 bp, Artificial Sequence
  Artificial sequence, Itgb3 mouse reverse primer
gtccccacagttacattg

SEQ ID NO: 62 - DNA, 20 bp, Artificial Sequence
  Artificial sequence, Ctsk mouse forward primer
agagagcagtggcgcgggta

SEQ ID NO: 63 - DNA, 21 bp, Artificial Sequence
  Artificial sequence, Ctsk mouse reverse primer
ccagctctctccccagctgtt

SEQ ID NO: 64 - DNA, 20 bp, Artificial Sequence
  Artificial sequence, Tm7sf4 mouse forward primer
tgggtgctgtttgccgctgt

SEQ ID NO: 65 - DNA, 23 bp, Artificial Sequence
  Artificial sequence, Tm7sf4 reverse primer
tgggttccttgcttctctccacg

SEQ ID NO: 66 - DNA, 20 bp, Artificial Sequence
  Artificial sequence, Jundam2 mouse forward primer
tgcgcccttgcacttcctgg

SEQ ID NO: 67 - DNA, 20 bp, Artificial Sequence
  Artificial sequence, Jundam2 mouse reverse primer
gccgctctgactccctctgc

SEQ ID NO: 68 - DNA, 23 bp, Artificial Sequence
  Artificial sequence, Gapdh mouse forward primer
tcccactcttccaccttcgatgc

SEQ ID NO: 69 - DNA, 24 bp, Artificial Sequence
  Artificial sequence, Gapdh mouse reverse primer
gggtctgggatggaaattgtgagg

SEQ ID NO: 70 - DNA, 22 bp, Artificial Sequence
  Artificial sequence, B-actin forward primer
tcctcctgagcgcaagtactct

SEQ ID NO: 71 - DNA, 22 bp, Artificial Sequence
  Artificial sequence, B-actin mouse reverse primer
cggactcatcgtactcctgctt

SEQ ID NO: 72 - DNA, 21 bp, Artificial Sequence
  Artificial construct, shRNA #1 sense, 595-615
aaggtgtgtggggcctcgggt

SEQ ID NO: 73 - DNA, 21 bp, Artificial Sequence
  Artificial construct, shRNA #1 antisense 595-615
acccgaggccccacacacctt

SEQ ID NO: 74 - DNA, 136 bp, Artificial Sequence
  Artificial construct, shRNA #1 full construct
gatctccaaggtgtgtggggcctcgggtttcaagagaacccgaggccccacacacctttttttggaaaagcttttccaaaaaaaggtgtgtggggcctcgggttctcttgaaacccgaggccccacacaccttgga

SEQ ID NO: 75 - DNA, 19 bp, Artificial Sequence
  Artificial construct, shRNA #2, 3407 oligo 47
cctttaacaaggagatgat

SEQ ID NO: 76 - DNA, 19 bp, Artificial Sequence
  Artificial construct, shRNA #2 antisense 3407 at oligo 47
atcatctccttgttaaagg

SEQ ID NO: 77 - DNA, 128 bp, Artificial Sequence
  Artificial construct, shRNA #2 full construct
gatctcccctttaacaaggagatgatttcaagagaatcatctccttgttaaaggtttttggaaaagcttttccaaaaacctttaacaaggagatgattctcttgaaatcatctccttgttaaagggga

SEQ ID NO: 78 - DNA, 19 bp, Artificial Sequence
  Artificial construct, shRNA #3 sense strand 4574 at oligo 71
cgtacaagtagttctgtat

SEQ ID NO: 79 - DNA, 19 bp, Artificial Sequence
  Artificial construct, shRNA #3 antisense strand 4574 at oligo 71
atacagaactacttgtacg

SEQ ID NO: 80 - DNA, 128 bp, Artificial Sequence
  Artificial construct, shRNA #3 full construct
gatctcccgtacaagtagttctgtatttcaagagaatacagaactacttgtacgtttttggaaaagcttttccaaaaacgtacaagtagttctgtattctcttgaaatacagaactacttgtacggga

SEQ ID NO: 81 - DNA, 19 bp, Artificial Sequence
  Artificial construct, shRNA #4 sense strand 5005 at oligo 74
cccagaatactgatggaat

SEQ ID NO: 82 - DNA, 19 bp, Artificial Sequence
  Artificial construct, shRNA #4 antisense 5005 at oligo 74
attccatcagtattctggg

SEQ ID NO: 83 - DNA, 128 bp, Artificial Sequence
  Artificial construct, shRNA #4 full construct
gatctcccccagaatactgatggaatttcaagagaattccatcagtattctgggtttttggaaaagcttttccaaaaacccagaatactgatggaattctcttgaaattccatcagtattctggggga

SEQ ID NO: 84 - DNA, 19 bp, Artificial Sequence
  Artificial construct, shRNA #5 sense strand 5411 at oligo 83
gctagttgaatacttgaat

SEQ ID NO: 85 - DNA, 19 bp, Artificial Sequence
  Artificial construct, shRNA #5 antisense strand 5411 at oligo 83
gttcaagtattcaactagc

SEQ ID NO: 86 - DNA, 128 bp, Artificial Sequence
  Artificial construct, shRNA #5 full construct
gatctccgctagttgaatacttgaatttcaagagagttcaagtattcaactagctttttggaaaagcttttccaaaaagctagttgaatacttgaactctcttgaaattcaagtattcaactagcgga

SEQ ID NO: 87 - DNA, 19 bp, Artificial Sequence
  Artificial construct, shRNA #6 sense strand 5440 at oligo 84
ccagtaagatcactgttta

SEQ ID NO: 88 - DNA, 19 bp, Artificial Sequence
  Artificial construct, shRNA #6 antisense strand 5440 at oligo 84
taaacagtgatcttactgg

SEQ ID NO: 89 - DNA, 128 bp, Artificial Sequence
  Artificial construct, shRNA #6 full construct
gatctccccagtaagatcactgtttattcaagagataaacagtgatcttactggtttttggaaaagcttttccaaaaaccagtaagatcactgtttatctcttgaataaacagtgatcttactgggga

SEQ ID NO: 90 - DNA, 19 bp, Artificial Sequence
  Artificial construct, shRNA #1 sense strand 4999 at oligo 76
ggagtattctcataagcta

SEQ ID NO: 91 - DNA, 19 bp, Artificial Sequence
  Artificial construct, shRNA #1 antisense strand 4999 at oligo 76
tagcttatgagaatactcc

SEQ ID NO: 92 - DNA, 128 bp, Artificial Sequence
  Artificial construct, shRNA#1 full construct mJagged1
gatctccggagtattctcataagctattcaagagatagcttatgagaatactcctttttggaaaagcttttccaaaaaggagtattctcataagctatctcttgaatagcttatgagaatactccgga

SEQ ID NO: 93 - DNA, 19 bp, Artificial Sequence
  Artificial construct, shRNA #2 sense strand 5014 at oligo 77
gctagttgaatacttgaat

SEQ ID NO: 94 - DNA, 19 bp, Artificial Sequence
  Artificial construct, shRNA #2 antisense strand 5014 at oligo 77
gttcaagtattcaactagc

SEQ ID NO: 95 - DNA, 128 bp, Artificial Sequence; identical to SEQ ID NO: 86
  Artificial construct, shRNA #2 full construct mJagged1

SEQ ID NO: 96 - DNA, 19 bp, Artificial Sequence
  Artificial construct, shRNA #3 sense strand at oligo 78
ccagttagatcactgttta

SEQ ID NO: 97 - DNA, 19 bp, Artificial Sequence
  Artificial construct, shRNA #3 antisense strand 5043 at oligo 78
taaacagtgatctaactgg

SEQ ID NO: 98 - DNA, 128 bp, Artificial Sequence
  Artificial construct, shRNA #3 full construct mJagged1
gatctccccagttagatcactgtttattcaagagataaacagtgatctaactggtttttggaaaagcttttccaaaaaccagttagatcactgtttatctcttgaataaacagtgatctaactgggga

SEQ ID NO: 99 - DNA, 19 bp, Artificial Sequence
  Artificial construct, shRNA #4 sense strand 4805 at oligo 70
ggaacagactgagctatat

SEQ ID NO: 100 - DNA, 19 bp, Artificial Sequence
  Artificial construct, shRNA #4 antisense strand 4805 at oligo 70
atatagctcagtctgttcc

SEQ ID NO: 101 - DNA, 128 bp, Artificial Sequence
  Artificial construct - shRNA #4 full construct mJagged1
gatctccggaacagactgagctatatttcaagagaatatagctcagtctgttcctttttggaaaagcttttccaaaaaggaacagactgagctatattctcttgaaatatagctcagtctgttccgga

SEQ ID NO: 102 - DNA, 19 bp, Artificial Sequence
  Artificial construct - shRNA #2 sense strand2 5014 at oligo 77
gctagttgaatacttgaac

SEQ ID NO: 103 - DNA, 19 bp, Artificial Sequence
  Artificial construct, shRNA #2 antisense strand2 5014 at oligo 77
attcaagtattcaactagc

SEQ ID NO: 104 - DNA, 19 bp, Artificial Sequence
  Artificial construct, sense strand 4629 at oligo 65
ccttgatagcatcacttta

SEQ ID NO: 105 - DNA, 19 bp, Artificial Sequence
  Artificial construct, antisense strand 4629 at oligo 65
taaagtgatgctatcaagg

SEQ ID NO: 106 - DNA, 19 bp, Artificial Sequence
  Artificial construct, sense strand 5086 at oligo 79
gccttaagtgaggaaatta

SEQ ID NO: 107 - DNA, 19 bp, Artificial Sequence
  Artificial construct, antisense strand 5086 at oligo 79
tgatttcctcacttaaggc